(19) United States
(12) Patent Application Publication
(10) Pub. No.: US 2015/0299798 A1
(43) Pub. Date: Oct. 22, 2015

(54) METHOD FOR CLASSIFICATION OF LIVER SAMPLES AND DIAGNOSIS OF FOCAL NODULE DYSPLASIA, HEPATOCELLULAR ADENOMA, AND HEPATOCELLULAR CARCINOMA

(71) Applicants: INTEGRAGEN, Evry (FR); INSTITUT NATIONAL DE LA SANTE ET DE LA RECHERCHE MEDICALE (INSERM), Paris (FR); UNIVERSITE PARIS DESCARTES, Paris (FR)

(72) Inventors: Aurelien De Reynies, Boulogne-Billancourt (FR); Pierre Laurent-Puig, Meudon (FR); Jessica Zucman-Rossi, Paris (FR); Jean-Charles Nault, Paris (FR)

(21) Appl. No.: 14/429,428

(22) PCT Filed: Sep. 23, 2013
(86) PCT No.: PCT/EP2013/069751

§ 371 Date: Mar. 19, 2015

Related U.S. Application Data
(60) Provisional application No. 61/704,383, filed on Sep. 21, 2012.

(30) Foreign Application Priority Data
Sep. 21, 2012 (EP) 12306145.9

(51) Int. Cl. C12Q 1/68; G06F 19/00; G06F 19/20

(52) U.S. Cl. CPC C12Q 1/6886; G06F 19/20; G06F 19/345; C12Q 2600/16; C12Q 2600/158; C12Q 2600/112

(57) ABSTRACT

The present invention relates to the technical field of liver diseases, their classification and diagnosis. It provides a new method for classifying a liver sample between non-hepatocellular sample; hepatocellular carcinoma (HCC) sample with further classification into one of subgroups G1 to G6; focal nodule dysplasia (FNH) sample; hepatocellular adenoma (HCA) sample with further classification into HNF1A mutated HCA, inflammatory HCA, β catenin mutated HCA or other HCA sample; and other benign liver sample, based on determination in vitro of genes expression profiles and analysis of the expression profile using algorithms calibrated with reference samples. The invention also provides kits for the classification of liver samples, and methods of treatment of liver disease in a subject based on a preliminary classification of a liver sample of said subject.

### TECHNICAL FIELD OF THE INVENTION

The present invention relates to the technical field of liver diseases, their classification and diagnosis. It provides a new method for classifying a liver sample between non-hepatocellular sample; hepatocellular carcinoma (HCC) sample with further classification into one of subgroups G1 to G6; focal nodule dysplasia (FNH) sample; hepatocellular adenoma (HCA) sample with further classification into HNF1A mutated HCA, inflammatory HCA, β catenin mutated HCA or other HCA sample; and other benign liver sample, based on determination in vitro of genes expression profiles and analysis of the expression profile using algorithms calibrated with reference samples. The invention also provides kits for the classification of liver samples, and methods of treatment of liver disease in a subject based on a preliminary classification of a liver sample of said subject.

### BACKGROUND ART

Hepatocellular carcinoma (HCC) represents one of the leading worldwide causes of death by cancer (El Serag H NEJM 2011). Despite the widespread use of imaging/non-invasive criteria for the diagnosis of HCC developed on cirrhosis, the differential diagnosis between HCC and others liver tumors remains difficult, even for an expert pathologist (international consensus group 2009). In this setting, regenerative and dysplastic macronodule, cholangiocarcinoma or metastasis of cancers of other tissue origin constitute classical pitfalls (Forner A Lancet 2012). Moreover, non-invasive criteria have not been validated for the diagnosis of HCC developed in non-cirrhotic liver contributing for 10% of the cases in western countries and more than 20% in eastern countries (Forner A Hepatology 2008). In this setting, tumor biopsy is mandatory and differential diagnosis with benign hepatocellular tumors (focal nodular hyperplasia, FNH and hepatocellular adenoma, HCA) could be challenging, especially between very well differentiated HCC and HCA (Bioulac-Sage P, sem liv dis 2011). Moreover, HCA constitute a heterogeneous group of benign liver tumors and a genotype/phenotype classification related to prognosis was recently identified (Zucman Rossi J Hepatology 2006; Van aalten S M J hepatol 2011). Four groups of HCA (HNF1A mutated, β catenin mutated, inflammatory and unclassified hepatocellular adenomas) were described and HCA with mutation activating β catenin was associated with an increased risk of malignant transformation in HCC.

Therefore, benign and malignant hepatocellular tumors comprise various subgroups of tumors defined by specific phenotypic and molecular features, which leads to diagnosis pitfalls and difficulty to assess their prognosis.

There is thus a need for new tools that help clinicians and pathologists in clinical practice for reliably distinguishing between the various types of tissues that can be present in a liver sample (hepatocellular or not; if hepatocellular, benign or malignant; if benign hepatocellular, focal nodule hyperplasia, hepatocellular adenoma, or none of both; if hepatocellular adenoma, which type of it), and thus to reliably classify liver samples taken from subjects suspected to suffer from a liver tumor.

Indeed, depending on the classification of the liver sample and thus on the final diagnosis, the patient will not be given the same treatment:
In case of benign focal nodule hyperplasia (FNH), therapeutic abstention without follow up is recommended;
In case of benign hepatocellular adenoma (HCA), usual treatments include surgical resection or therapeutic abstention with follow up. The selection of the best treatment may also depend on the more precise classification of HCA into HNF1A mutated, inflammatory, and β catenin mutated HCA. For instance, if the sample is diagnosed as HNF1A mutated HCA smaller than 5 cm, a follow up with imaging/clinical follow up only may be particularly useful, because of the low risk of hemorrhage and malignant transformation. If the sample is diagnosed as HNF1A mutated HCA with a size of more than 5 cm, a treatment with surgical resection may be particularly useful, because of the risk of hemorrhage. If the sample is diagnosed as inflammatory HCA with a size of less than 5 cm then a follow up with imaging/clinical follow up only may be particularly useful, because of the low risk of hemorrhage and malignant transformation. If the sample is diagnosed as Inflammatory HCA with a size of more than 5 cm, then a treatment with surgical resection, may be particularly useful, because of the risk of hemorrhage. If the sample is diagnosed as β catenin mutated HCA whatever the size, then a curative treatment with surgical resection may be particularly useful, because of the high risk of malignant transformation.
In case of hepatocellular carcinoma (HCC), the first treatment generally consists in tumor surgical resection, although alternative treatment may be used if tumor surgical resection is not possible. In addition, various adjuvant therapies may be administered after tumor surgical resection. Such adjuvant therapies include cytotoxic chemotherapy (in particular doxorubicin or association of gemcitabine and oxaliplatine) and/or targeted therapy (in particular sorafenib). The selection of the best treatment strategy (including the use or not of adjuvant therapy) may depend on the more precise type of HCC (see classification of HCC into one of subgroups G1 to G6 described in WO2007/063118A1) and/or on the prognosis of the patient. In particular, in case of bad prognosis, adjuvant therapy is generally given, while it is not systematically the case if the prognosis is good. In addition, if the liver sample has been further classified as HCC subgroup G1, then a treatment with IGFR1 inhibitor may be particularly useful, because of the activation of insulin growth factor pathway. If the liver sample has been further classified as HCC subgroup G1 or G2, then a treatment with Akt/mtor inhibitor may be particularly useful, because the activation of akt/mtor pathway. If the liver sample has been further classified as HCC subgroup G3, then a treatment with proteasome inhibitor may be particularly useful, because of the dysregulation of cell/cycle genes. If the liver sample has been further classified as HCC subgroup G5 or G6, then a treatment with Wnt inhibitor may be particularly useful, because of activation of Wnt/catenin pathway.

In this setting, a simple classification/diagnosis tool based on molecular profiling of a subject's liver sample would be very helpful.

Several genes have been associated to the classification of liver samples or the diagnosis of particular liver diseases. For instance, genes differentially expressed in hepatocellular and non-hepatocellular tissue have been described in Odom et al-2004. Genes associated to benign or malignant hepatocellular tumors have been identified in Llovet et al-2006, Capurro et al-2003, Chuma et al-2003, Tsunedomi et al-2005 and Kondoh et al-1999. Genes differentially expressed in focal nodule hyperplasia (FNH) have been disclosed in Rebouissou et al-2008 and Paradis et al-2003. Genes differentially expressed in HNF1A mutated HCA have been disclosed in Rebouissou et al-2007 and Bioulac Sage et al-2007. Genes associated to β catenin mutations have been described in Boyault et al-2007, Bioulac Sage et al-2007, Cadoret et al-2002, Yamamoto et al-2005, Benhamouche et al-2006, and Rebouissou et al-2008. Genes differentially expressed in inflammatory HCA have been disclosed in Rebouissou et al-2009 and Bioulac Sage et al-2007.

However, there has been no disclosure in the prior art of a true method permitting to simply and reliably classify a liver sample among the various types of liver diseases, and to simply and reliably diagnose the presence of non-hepatocellular tissue in liver, malign hepatocellular carcinoma (HCC), benign focal nodule hyperplasia (FNH), hepatocellular adenoma and its subtypes.

Based on a new strategy of analysis of microarray and quantitative PCR data obtained from various types of liver samples, the inventors have constructed a simple and reliable molecular algorithm for the precise classification and diagnosis of liver samples. In particular, the inventors have established several signatures able:
To reliably distinguish between hepatocellular and non-hepatocellular sample (metastasis of other tissue origin, cholangiocarcinoma), or between benign and malignant (hepatocellular carcinoma) hepatocellular samples;
To precisely diagnose, among benign hepatocellular samples the presence of focal nodule hyperplasia (FNH) or hepatocellular adenoma (HCA); and
To precisely diagnose, among HCA samples, the type of HCA sample: HNF1A mutated HCA, inflammatory HCA, β catenin mutated HCA, or other HCA.

A global set of 55 genes permits to reliably classify a liver between all those types of liver samples.

### DESCRIPTION OF THE INVENTION

The present invention thus relates to a method for classifying in vitro a liver sample as a non-hepatocellular sample, a hepatocellular carcinoma (HCC) sample, a focal nodule dysplasia (FNH) sample, a hepatocellular adenoma (HCA) sample or another benign liver sample, comprising:
a) Determining in vitro from said liver sample an expression profile comprising or consisting of the 38 following genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9, and optionally one or more internal control genes, or an Equivalent Expression Profile thereof;
b) Determining if said liver sample is a hepatocellular or a non-hepatocellular sample, based on the expression levels measured for an expression profile comprising or consisting of the 9 following genes: EPCAM, HNF4A, CYP3A7, FABP1, HAL, AFP, GNMT, TFRC, and C8A, and optionally one or more internal control genes, or an Equivalent Expression Profile thereof, using at least one algorithm calibrated with at least one reference liver sample;
c) If said liver sample is a hepatocellular sample, then determining if said hepatocellular sample is a HCC sample or a benign hepatocellular sample, based on the expression levels measured for an expression profile comprising or consisting of the 9 following genes: AFP, CAP2, LCAT, ANGPT2, AURKA, CDC20, DHRS2, LYVE1, and ADM, and optionally one or more internal control genes, or an Equivalent Expression Profile thereof, using at least one algorithm calibrated with at least one reference liver sample;
d) If said liver sample is a benign hepatocellular sample, then determining if said benign hepatocellular sample is a FNH sample, based on the expression levels measured for an expression profile comprising or consisting of the 13 following genes: HAL, ANGPTL7, GLUL, ANGPT1, HMGB3, GMNN, RAMP3, RHBG, UGT2B7, LGR5, RARRES2, RBM47, and GIMAP5, and optionally one or more internal control genes, or an Equivalent Expression Profile thereof, using at least one algorithm calibrated with at least one reference liver sample;
e) If said liver sample is a benign hepatocellular sample, then determining if said benign hepatocellular sample is a HCA sample, based on the expression levels measured for an expression profile comprising or consisting of the 13 following genes: HAL, CYP3A7, LCAT, LYVE1, AKR1B10, GLS2, KRT19, ESR1, SDS, MERTK, EPHA1, CCL5, and CYP2C9, and optionally one or more internal control genes, or an Equivalent Expression Profile thereof, using at least one algorithm calibrated with at least one reference liver sample;
f) If said benign hepatocellular sample is neither a FNH sample nor a HCA sample, then it is classified as another benign liver sample.

In an advantageous embodiment, the method according to the invention further comprises, if the liver sample is diagnosed as a HCA sample, classifying said HCA sample into one of the following HCA subgroups: HNF1A mutated HCA, inflammatory HCA, β catenin mutated HCA or other HCA, by:
a) Further determining in vitro from said HCA sample an expression profile comprising or consisting of the 8 additional following genes: HAMP, SAA2, NRCAM, REG3A, AMACR, TAF9, LAPTM4B, and IGF2BP3;
b) Determining if said HCA sample is or not a HNF1A mutated HCA sample, based on the expression levels measured for an expression profile comprising or consisting of the 4 following genes: FABP1, ANGPT2, DHRS2, and UGT2B7, and optionally one or more internal control genes, or an Equivalent Expression Profile thereof, using at least one algorithm calibrated with at least one reference liver sample;
c) Determining if said HCA sample is or not an inflammatory HCA sample, based on the expression levels measured for an expression profile comprising or consisting of the 7 following genes: ANGPT2, GLS2, EPHA1, CCI5, HAMP, SAA2, and NRCAM, and optionally one or more internal control genes, or an Equivalent Expression Profile thereof, using at least one algorithm calibrated with at least one reference liver sample;
d) Determining if said HCA sample is or not a β catenin mutated HCA sample, based on the expression levels measured for an expression profile comprising or consisting of the 13 following genes: TFRC, HAL, CAP2, GLUL, HMGB3, LGR5, GIMAP5, AKR1B10, REG3A, AMACR, TAF9, LAPTM4B, and IGF2BP3, and optionally one or more internal control genes, or an Equivalent Expression Profile thereof, using at least one algorithm calibrated with at least one reference liver sample;
e) If said HCA sample is neither a HNF1A mutated HCA sample, an inflammatory HCA sample, nor a β catenin mutated HCA sample, then it is classified as another HCA sample.

In another advantageous embodiment, the method according to the invention further comprises, if the liver sample is diagnosed as a HCC sample, classifying said HCC sample into one of subgroups G1 to G6 defined by the clinical and genetic main features described in following Table 1:

| | G1 | G2 | G3 | G4 | G5 | G6 |
|---|---|---|---|---|---|---|
| Chromosome instability | + | + | + | − | − | − |
| Early relapse and death | + | + | + | − | − | − |
| TP53 mutation | − | + | + | − | − | − |
| HBV infection | + | + | − | − | − | − |
| Low copy number | + | − | − | − | − | − |
| High copy number | − | + | − | − | − | − |
| CTNNB1 mutation | − | − | − | − | + | + |
| Satellite nodules | − | − | − | − | − | + |

wherein classification is made by:
a) Further determining in vitro from said HCC sample an expression profile comprising or consisting of the 11 additional following genes: RAB1A, REG3A, NRAS, PIR, LAMA3, G0S2, HN1, PAK2, CDH2, HAMP, and SAE1; and
b) calculating 6 subgroup distances based on the expression levels measured for an expression profile comprising or consisting of the 16 following genes: RAB1A, REG3A, NRAS, RAMP3, MERTK, PIR, EPHA1, LAMA3, G0S2, HN1, PAK2, AFP, CYP2C9, CDH2, HAMP, and SAE1, and optionally one or more internal control genes, or an Equivalent Expression Profile thereof; and
c) classifying said HCC tumor in the subgroup for which the subgroup distance is the lowest.

Such classification of HCC samples into subgroups G1 to G6 has already been described in detailed in WO2007/063118A1, which content concerning such classification is herein incorporated by reference.

In a preferred embodiment, the HCC sample is classified into one of subgroups G1 to G6 using the following formula for calculating the distance of said HCC sample to each subgroup Gₖ, 1≦k≦6:

$${\sum }_{t = 1 … 16} \frac{\begin{matrix}Distance ( HCC sample , subgroup {G}_{k} ) = \\ {( Δ Ct ( HCC sample , subgroup {G}_{k} , {gene}_{t} ) - μ ( subgroup {G}_{k} , {gene}_{t} ) )}^{2}\end{matrix}}{σ ( {gene}_{t} )}$$

wherein for each geneₜ and subgroup Gₖ, the μ(subgroup Gₖ, geneₜ) and σ(geneₜ) values are the following:

| μ | G1 | G2 | G3 | G4 | G5 | G6 | σ |
|---|---|---|---|---|---|---|---|
| gene 1 (RAB1A) | −16.39 | −16.04 | −16.29 | −17.15 | −17.33 | −16.95 | 0.23 |
| gene 2 (PAP) | −28.75 | −27.02 | −23.48 | −27.87 | −19.23 | −11.33 | 16.63 |
| gene 3 (NRAS) | −16.92 | −17.41 | −16.25 | −17.31 | −16.96 | −17.26 | 0.27 |
| gene 4 (RAMP3) | −23.54 | −23.12 | −25.34 | −22.36 | −23.09 | −23.06 | 1.23 |
| gene 5 (MERTK) | −18.72 | −18.43 | −21.24 | −18.29 | −17.03 | −16.16 | 7.23 |
| gene 6 (PIR) | −18.44 | −19.81 | −16.73 | −18.28 | −17.09 | −17.25 | 0.48 |
| gene 7 (EPHA1) | −16.68 | −16.51 | −19.89 | −17.04 | −18.70 | −21.98 | 1.57 |
| gene 8 (LAMA3) | −20.58 | −20.44 | −20.19 | −21.99 | −18.77 | −16.85 | 2.55 |
| gene 9 (G0S2) | −14.82 | −17.45 | −18.18 | −14.78 | −17.99 | −16.06 | 3.88 |
| gene 10 (HN1) | −16.92 | −17.16 | −15.91 | −17.88 | −17.72 | −17.93 | 0.54 |
| gene 11 (PAK2) | −17.86 | −16.56 | −16.99 | −18.14 | −17.92 | −17.97 | 0.58 |
| gene 12 (AFP) | −16.68 | −12.36 | −26.80 | −27.28 | −25.97 | −23.47 | 14.80 |
| gene 13 (CYP2C9) | −18.27 | −16.99 | −16.26 | −16.23 | −13.27 | −14.44 | 5.47 |
| gene 14 (CDH2) | −15.20 | −14.76 | −18.91 | −15.60 | −15.48 | −17.32 | 10.59 |
| gene 15 (HAMP) | −19.53 | −20.19 | −21.32 | −18.51 | −25.06 | −26.10 | 13.08 |
| gene 16 (SAE1) | −17.37 | −17.10 | −16.79 | −18.22 | −17.72 | −18.16 | 0.31 |

In the above methods according to the invention, when a HCC sample is further classified into one of subgroups G1 to G6, or when a HCA sample is further classified as a HNF1A mutated HCA sample, an inflammatory HCA sample, or a β catenin mutated HCA sample, the two steps of determining in vitro the first expression profile for general classification and the second expression profile for further subgroup classification may be performed either simultaneously as only one step, or separately as two distinct steps. Preferably, they are performed simultaneously as only one step, since this is the simplest manner to do it.

In the above methods according to the invention, reference samples are used in order to calibrate an algorithm or a distance function, which may then be used to classify a new liver sample. In advantageous embodiments of the methods of the invention, reference samples used for calibrating algorithms or the distance function used for interpreting expression profiles are the following:
a) For determining if a liver sample is or not a hepatocellular sample: at least one (preferably several) hepatocellular sample and at least one (preferably several) non-hepatocellular sample;
b) For determining if a hepatocellular sample is or not a HCC sample: at least one (preferably several) benign sample and at least one (preferably several) HCC sample;
c) For determining if a benign hepatocellular sample is or not a FNH sample: at least one (preferably several) FNH sample and at least one (preferably several) non-FNH benign hepatocellular sample;
d) For determining if a benign hepatocellular sample is or not a HCA sample: at least one (preferably several) HCA sample and at least one (preferably several) non-HCA benign hepatocellular sample;
e) For determining if a HCA sample is or not a HNF1A mutated HCA sample: at least one (preferably several) HNF1A mutated HCA sample and at least one (preferably several) non-HNF1A mutated HCA sample;
f) For determining if a HCA sample is or not an inflammatory HCA sample: at least one (preferably several) inflammatory HCA sample and at least one (preferably several) non-inflammatory HCA sample;
g) For determining if a HCA sample is or not a β catenin mutated HCA sample: at least one (preferably several) β catenin mutated HCA sample and at least one (preferably several) non-β catenin mutated HCA sample; and
h) For classifying a HCC sample into one of subgroups G1 to G6: at least one (preferably several) sample of each G1 to G6 subgroups.

By “subject”, it is meant any human subject, regardless of sex or age.

By “liver sample”, it is meant any sample obtained by taking part of the liver of a subject. By “hepatocellular” liver sample, it is intended to mean that the liver sample analyzed is mainly made of hepatocytes or progenitors of hepatocytes, which may or not be transformed. Conversely, by “non-hepatocellular” liver sample, it is intended to mean that the liver sample is mainly made of cells others than hepatocytes or progenitors of hepatocytes. Non-hepatocellular liver samples notably include liver samples mainly made of metastases of cancers of non-hepatocellular origin (such as lung, breast, colon, or skin cancer for instance) and liver samples mainly made of cholangiocarcinoma, a cancer composed of mutated epithelial cells (or cells showing characteristics of epithelial differentiation) that originate in the bile ducts which drain bile from the liver into the small intestine. Cholangiocarcinoma thus occurs in the liver but is made of non-hepatocellular cells.

By “malignant hepatocellular samples”, “hepatocellular carcinoma” or “HCC”, it is intended to mean a primary malignancy of liver hepatocytes or hepatocytes progenitors. HCC is generally diagnosed by histological analysis, and is characterized by hepatocytes proliferation with an elevated nuclear to cytoplasmic ratio, trabecular architecture and atypical nuclei.

Benign hepatocellular samples include samples affected by FNH or HCA, and other benign hepatocellular samples. By “focal nodule hyperplasia” or “FNH”, it is intended to mean a benign tumor of the liver generally characterized by a central stellate scar seen in 60-70% of cases. Microscopically, a lobular proliferation of bland-appearing hepatocytes with a bile ductular proliferation and malformed vessels within the fibrous scar is the most common pattern. Other patterns include telangiectatic, hyperplastic-adenomatous, and lesions with focal large-cell dysplasia. It is generally diagnosed by histological analysis. By “hepatocellular adenoma”, “hepatic adenoma”, “hepadenoma” or “HCA”, it is intended to mean a benign liver tumor characterized by well-circumscribed nodules that consist of sheets of hepatocytes with a bubbly vacuolated cytoplasm. The hepatocytes are on a regular reticulin scaffold and less or equal to three cell thick. It is generally diagnosed by histological analysis. Subgroups of HCA include “HNF1A” mutated HCA”, which is a HCA characterized by the presence of mutation(s) in the HNF1A gene, “β catenin mutated HCA”, which is a HCA characterized by the presence of mutation(s) in the β catenin gene, “inflammatory HCA”, which is a HCA characterized by presence of inflammatory infiltrate, sinusoidal dilatation, dystrophic arteries and overexpression of SAA protein at histological and immunohistochemical analysis, and “other HCA”, which corresponds to a HCA sample that is neither a HNF1A” mutated HCA, a β catenin mutated HCA, nor an inflammatory HCA. Other benign hepatocellular samples include healthy liver samples, cirrhotic liver samples, and regenerative macronodule samples (with or without dysplasia). By “regenerative macronodule”, it is intended to mean liver nodules of more than 3 mm, which form in response to necrosis, altered circulation, or other stimuli, characterized by benign hepatocyte with or without cell dysplasia. It is generally diagnosed by histological analysis.

In the methods according to the invention, liver samples are analyzed. Such liver samples may notably be a liver biopsy or a partial or whole liver tumor surgical resection. Reference samples used for calibrating algorithms and distance function are also liver samples, preferably of the same type as those analyzed.

The above methods according to the invention are based on the in vitro determination of particular expression profiles comprising or consisting of specific genes. 55 genes are needed for performing the most complete classification (non-hepatocellular; HCC with further classification into one of subgroups G1 to G6; FNH; HCA with further classification into HNF1A mutated HCA, inflammatory HCA, β catenin mutated HCA or other HCA; and other benign liver sample). Information concerning those 55 genes is provided in Table 2 below:

TABLE 2
Description of the 55 genes included in the classification algorithm, as well as
genes considered as equivalents, i.e. the at most 10 genes which expression in HCC
samples is best correlated to the original gene, with a Pearson's correlation coefficient
≧0.3 or ≦−0.3.

| | | | | Equivalent genes |
|---|---|---|---|---|
| | | | | among the 103 genes |
| Gene short | | Chromosome | | tested in quantitative |
| name | HUGO Gene name | location | Biological functions | PCR |
| ADM | Adrenomedullin | 11p15.4 | Activation of | ANGPT2; CHKA; |
| | | | adrenomedullin pathway, | ENO1; G6PD; HN1; |
| | | | angiogenesis, | NPEPPS; RAN; TAF9 |
| | | | vasodilatation | |
| AFP | Alpha-fetoprotein | 4q11-q13 | Foetal liver gene, stem | CYP3A7; GPC3; HAL |
| | | | cell marker | |
| AKR1B10 | Aldo-keto reductase family | 7q33 | Reduction of aliphatic | ANGPTL7; CAP2; |
| | 1, member B10 (aldose | | and aromatic aldehydes | GPC3; PIR; SPP1; |
| | reductase) | | | TKT; |
| | | | | AKR1C1.AKR1C2 |
| AMACR | Alpha-methylacyl-CoA | 5p13.2-q11.1 | Fatty acid degradation, | GLUL; HAL; LAMA3; |
| | racemase | | peroxisomal beta- | MERTK; MIA3; MME; |
| | | | oxidation | PHB; PIR; REG3A; |
| | | | | SLC16A1; SLPI; |
| | | | | TBX3; |
| | | | | AKR1C1.AKR1C2; |
| | | | | HNF4A |
| ANGPT1 | Angiopoietin 1 | 8q23.1 | Vascular development | GIMAP5; KLRB1; |
| | | | and angiogenesis | RAMP3 |
| ANGPT2 | Angiopoietin 2 | 8p23 | Vascular development | BIRC5; CCNB1; |
| | | | and angiogenesis | CDC20; DPP8; |
| | | | | G6PD; GLA; HN1; |
| | | | | KPNA2; NEK7; |
| | | | | NEU1; NPEPPS; |
| | | | | NRAS; RAN; SAE1; |
| | | | | TRIP13; CKS2; |
| | | | | DLGAP5 |
| ANGPTL7 | Angiopoietin-like 7 | 1p36 | Vascular development | AKR1B10; ESR1; |
| | | | and angiogenesis | GPC3; SPP1; TKT |
| AURKA | Aurora kinase A | 20q13 | Cell cycle regulation, | BIRC5; CCNB1; |
| | | | chromosome segregation | CDC20; GLA; HN1; |
| | | | | HSPA4; KPNA2; |
| | | | | NRAS; SAE1; |
| | | | | TRIP13; CKS2; |
| | | | | RRM2; DLGAP5 |
| C8A | Complement component 8, | 1p32.2 | Component of the | CYP2C9; ESR1; |
| | alpha polypeptide | | complement system | FABP1; G6PD; |
| | | | | GNMT; LCAT; |
| | | | | RARRES2; SAE1; |
| | | | | UGT2B7; STEAP3; |
| | | | | SERPIN |
| CAP2 | CAP, adenylate cyclase- | 6p22.3 | Interaction with adenylyl | DPP8; HSPA4; MIA3; |
| | associated protein, 2 | | cyclase-associated | NEK7; NEU1; SAE1; |
| | (yeast) | | protein and actin | TAF9 |
| CCL5 | Chemokine (C-C motif) | 17q11.2-q12 | Immunoregulatory and | G6PD; GIMAP5; |
| | ligand 5 | | inflammatory processes | KLRB1; RAMP3 |
| CDC20 | Cell division cycle 20 | 1p34.1 | Cell cycle regulation, | AURKA; BIRC5; |
| | homolog (S. cerevisiae) | | | CCNB1; G6PD; GLA; |
| | | | | HN1; KPNA2; NRAS; |
| | | | | SAE1; TRIP13; |
| | | | | CKS2; RRM2; |
| | | | | DLGAP5 |
| CDH2 | Cadherin 2, type 1, N- | 18q12.1 | Calcium dependent cell | MIA3; |
| | cadherin | | adhesion protein | AKR1C1.AKR1C2; |
| | (neuronal) | | | HNF1A |
| CYP2C9 | Cytochrome P450, family 2, | 10q24.1 | Drug metabolism and | FABP1; GNMT; |
| | subfamily C, polypeptide 9 | | synthesis of cholesterol | LCAT; RARRES2; |
| | | | and steroids. | RHBG; UGT2B7; |
| | | | | CKS2; C8A; |
| | | | | AKR1C1.AKR1C2; |
| | | | | SERPIN |
| CYP3A7 | Cytochrome P450, family | 7q21-q22.1 | Drug and aflatoxin B1 | AFP; CRP; CYP2C9; |
| | 3, | | metabolism, synthesis of | EPHA1; FABP1; |
| | subfamily A, polypeptide 7 | | cholesterol and steroids. | GLS2; GPC3; HAL; |
| | | | | SLPI |
| DHRS2 | Dehydrogenase/reductase | 14q11.2 | NADPH-dependent | AMACR; AURKA; |
| | (SDR family) member 2 | | dicarbonyl reductase | BIRC5; CAP2; |
| | | | activity | CCNB1; CHKA; |
| | | | | GLUL; HAMP; |
| | | | | HSPA4; MIA3; PIR; |
| | | | | SLC16A1; TAF9; |
| | | | | TBX3; RRM2; |
| | | | | AKR1C1.AKR1C2 |
| EPCAM | Epithelial cell adhesion | 2p21 | Membrane protein and | HN1; NPEPPS; NTS; |
| | molecule | | liver stem cell marker | RARRES2; TBX3; |
| | | | | C8A; KRT19; |
| | | | | AKR1C1.AKR1C2 |
| EPHA1 | EPH receptor A1 | 7q32-q36 | Ephrin receptor subfamily | CYP3A7; GLS2; |
| | | | of the protein-tyrosine | GLUL; HAL; REG3A; |
| | | | kinase family | SLPI; STEAP3; |
| | | | | RBM47 |
| ESR1 | Estrogen receptor 1 | 6q24-q27 | Estrogen binding, DNA | AURKA; BIRC5; |
| | | | binding, and activation of | CCNB1; CDC20; |
| | | | transcription | TRIP13; CKS2; |
| | | | | RRM2 |
| FABP1 | Fatty acid binding protein | 2p11 | Binds free fatty acids and | CRP; CYP2C9; |
| | 1, liver | | their coenzyme A | CYP3A7; GNMT; |
| | | | derivatives | HAL; LCAT; UGT2B7; |
| | | | | C8A; HNF4A; |
| | | | | SERPIN |
| G0S2 | G0/G1 switch 2 | 1q32.2-q41 | Pro-apoptotic factor | CXCR7; LGR5 |
| GIMAP5 | GTPase, IMAP family | 7q36.1 | GTP-binding superfamily, | ANGPT1; CCL5; |
| | member 5 | | mitochondrial integrity | KLRB1; RAMP3; |
| | | | | LYVE1 |
| GLS2 | Glutaminase 2 (liver, | 12q13 | Regulation of glutamine | CAP2; EPHA1; GLUL; |
| | mitochondrial) | | catabolism, mitochondrial | GNMT; HAL; LAMA3; |
| | | | respiration | SDS; SLPI; STEAP3; |
| | | | | CYP2C19 |
| GLUL | Glutamate-ammonia ligase | 1q31 | Synthesis of glutamine | AMACR; CAP2; |
| | | | | GLS2; GLUL; HAL; |
| | | | | LAMA3; LGR5; |
| | | | | REG3A; SLPI; TBX3 |
| GMNN | Geminin, DNA replication | 6p21.32 | Inhibition of DNA | ARFGEF2; AURKA; |
| | inhibitor | | replication, cell cycle | BIRC5; CCNB1; |
| | | | regulation | CDC20; DPP8; |
| | | | | G6PD; GLA; HN1; |
| | | | | HSPA4; KPNA2; |
| | | | | NEK7; NEU1; NRAS; |
| | | | | PSMD1; RAN; SAE1; |
| | | | | TAF9; TRIP13; CKS2; |
| | | | | RRM2; DLGAP5 |
| GNMT | Glycine N- | 6p12 | Metabolism of methionine | CYP2C9; FABP1; |
| | methyltransferase | | | G6PD; GLS2; HN1; |
| | | | | LCAT; RARRES2; |
| | | | | UGT2B7; CKS2; C8A |
| HAL | Histidine ammonia-lyase | 12q22-q24.1 | Histidine catabolism | AFP; AMACR; CRP; |
| | | | | CYP3A7; EPHA1; |
| | | | | GLS2; GLUL; LAMA3; |
| | | | | REG3A; SDS; SLPI; |
| | | | | TBX3 |
| HAMP | Hepcidin antimicrobial | 19q13.1 | Iron homeostasis, | AURKA; BIRC5; |
| | peptide | | inflammation target gene | CAP2; CCNB1; |
| | | | | CDC20; CRP; ESR1; |
| | | | | HSPA4; LCAT; NEK7; |
| | | | | SAA2; SAE1; TFRC; |
| | | | | TRIP13; CKS2; |
| | | | | STEAP3; RRM2; |
| | | | | DLGAP5; LYVE1 |
| HMGB3 | High mobility group box 3 | Xq28 | DNA replication, | ARFGEF2; AURKA; |
| | | | nucleosome assembly | BIRC5; C14orf156; |
| | | | and transcription | CCNB1; CDC20; |
| | | | | GLA; HN1; HSPA4; |
| | | | | KPNA2; LAPTM4B; |
| | | | | NRAS; RAN; SAE1; |
| | | | | SLPI; TAF9; TFRC; |
| | | | | TRIP13; CKS2; |
| | | | | RRM2; DLGAP5; |
| | | | | RBM47 |
| HN1 | Hematological and | 17q25.1 | Regulation of androgen | AURKA; BIRC5; |
| | neurological | | receptor | CCNB1; CDC20; |
| | expressed 1 | | | ENO1; G6PD; GLA; |
| | | | | HSPA4; KPNA2; |
| | | | | NRAS; PDCD2; RAN; |
| | | | | SAE1; TRIP13; |
| | | | | CKS2; RRM2; |
| | | | | DLGAP5 |
| HNF4A | hepatocyte nuclear factor | 20q13.12 | Transcription factor, liver | AMACR; CYP2C9; |
| | 4, alpha | | development | FABP1; UGT2B7; |
| | | | | C8A; |
| | | | | AKR1C1.AKR1C2; |
| | | | | HNF1A; SERPIN |
| IGF2BP3 | Insulin-like growth factor 2 | 7p15.3 | Translation repression of | BIRC5; CCNB1; |
| | mRNA binding protein 3 | | insulin-like growth factor II | CDC20; G6PD; HN1; |
| | | | | KPNA2; NRAS; |
| | | | | SAE1; TRIP13; |
| | | | | CKS2; RRM2 |
| KRT19 | Keratin 19 | 17q21-q23 | Structural integrity of | CYP2C9; GNMT; |
| | | | epithelial cells, liver stem | HN1; IGF2BP3; |
| | | | cell marker | NPEPPS; NTS; |
| | | | | RARRES2; TBX3; |
| | | | | C8A; EPCAM; |
| | | | | AKR1C1.AKR1C2 |
| LAMA3 | Laminin, alpha 3 | 18q11.2 | Cell adhesion and | AMACR; BIRC5; |
| | | | migration | CAP2; CCNB1; |
| | | | | CDC20; CHKA; |
| | | | | DPP8; G6PD; GLA; |
| | | | | GLS2; GLUL; HAL; |
| | | | | HN1; HSPA4; LGR5; |
| | | | | NEK7; NPEPPS; |
| | | | | NRAS; PSMD1; |
| | | | | REG3A; SAE1; TAF9; |
| | | | | TBX3; TKT; TRIP13 |
| LAPTM4B | Lysosomal protein | 8q22.1 | Regulation of apoptosis | AURKA; BIRC5; |
| | transmembrane | | and lysosomal | CCNB1; CDC20; |
| | 4 beta | | degradation | G6PD; HN1; HSPA4; |
| | | | | KPNA2; NRAS; RAN; |
| | | | | SAE1; TRIP13; |
| | | | | CKS2; DLGAP5 |
| LCAT | Lecithin-cholesterol | 16q22.1 | Extracellular metabolism | BIRC5; CCNB1; |
| | acyltransferase | | of plasma lipoproteins | CDC20; ESR1; |
| | | | | G6PD; GLA; GNMT; |
| | | | | HN1; NPEPPS; |
| | | | | SPP1; TRIP13; |
| | | | | CKS2; RRM2; C8A |
| LGR5 | Leucine-rich repeat | 12q22-q23 | Wnt/catenin signaling | AMACR; ANGPTL7; |
| | containing | | | CHKA; G0S2; GLS2; |
| | G protein-coupled | | | GLUL; HAL; LAMA3; |
| | receptor 5 | | | MERTK; REG3A; |
| | | | | RHBG; SDS; SLPI; |
| | | | | TBX3 |
| LYVE1 | Lymphatic vessel | 11p15 | Autocrine regulation of | AURKA; BIRC5; |
| | endothelial | | cell growth, metastasis | CCNB1; CDC20; |
| | hyaluronan receptor 1 | | | ESR1; HAMP; SAA2; |
| | | | | TRIP13; RRM2 |
| MERTK | C-mer proto-oncogene | 2q14.1 | Member of the | AMACR; CAP2; CRP; |
| | tyrosine kinase | | MER/AXL/TYRO3 | GLS2; GLUL; HAL; |
| | | | receptor kinase family | LAMA3; LGR5; MME; |
| | | | | NRAS; PSMD1; |
| | | | | SLC16A1; TAF9 |
| NRAS | Neuroblastoma RAS viral | 1p13.2 | Oncogene, activation of | ARFGEF2; AURKA; |
| | (v-ras) | | MAP kinase pathway | BIRC5; CCNB1; |
| | oncogene homolog | | | CDC20; DPP8; |
| | | | | ENO1; G6PD; GLA; |
| | | | | HN1; HSPA4; |
| | | | | KIAA0090; KPNA2; |
| | | | | PDCD2; PSMD1; |
| | | | | RAN; SAE1; TAF9; |
| | | | | TRIP13 |
| NRCAM | Neuronal cell adhesion | 7q31 | Cell adhesion molecule, | CRP; G6PD; GNMT; |
| | molecule | | cell migration | HN1; IGF2BP3; SPP1 |
| PAK2 | p21 protein (Cdc42/Rac)- | 3q29 | Control of cell survival | AGPS; ARFGEF2; |
| | activated | | and growth. Modulation of | AURKA; BIRC5; |
| | kinase 2 | | apoptosis. | C14orf156; CCNB1; |
| | | | | DPP8; ENO1; G6PD; |
| | | | | GLA; HN1; HSPA4; |
| | | | | KPNA2; NEK7; |
| | | | | NEU1; NRAS; |
| | | | | PDCD2; PSMD1; |
| | | | | RAN; SAE1; TAF9; |
| | | | | TKT |
| PIR | Pirin (iron-binding nuclear | Xp22.31 | Transcriptional | AKR1B10; AMACR; |
| | protein) | | coregulator, involve in | AURKA; C14orf156; |
| | | | apoptosis and cell | CAP2; CCNB1; |
| | | | migration | ENO1; GLA; GLUL; |
| | | | | HSPA4; KPNA2; |
| | | | | MIA3; NUDT9; |
| | | | | PSMD1; RRAGD; |
| | | | | SLC16A1; TAF9; |
| | | | | TBX3; TKT; |
| | | | | AKR1C1.AKR1C2 |
| RAB1A | RAB1A, member RAS | 2p14 | Ras superfamily of | AGPS; ARFGEF2; |
| | oncogene family | | GTPases, transit of | C14orf156; DPP8; |
| | | | protein through Golgi | ENO1; G6PD; GLA; |
| | | | compartment | HN1; HSPA4; |
| | | | | KIAA0090; KPNA2; |
| | | | | NEK7; NEU1; NRAS; |
| | | | | NUDT9; PAK2; |
| | | | | PDCD2; PSMD1; |
| | | | | RAN; SAE1; TAF9; |
| | | | | TFRC |
| RAMP3 | Receptor (G protein- | 7p13-p12 | Adrenomedullin receptor, | ANGPT1; BIRC5; |
| | coupled) | | vasodilatation, | CCL5; CCNB1; |
| | activity modifying protein 3 | | angiogenesis | CYP2C9; ESR1; |
| | | | | GIMAP5; GNMT; |
| | | | | HAMP; KLRB1; |
| | | | | LCAT; SDS; UGT2B7; |
| | | | | CKS2; STEAP3; |
| | | | | RRM2; CYP2C19; |
| | | | | C8A |
| RAN | RAN, member RAS | 12q24.3 | Ras/raf pathway, control | C14orf156; CCNB1; |
| | oncogene family | | of | DPP8; ENO1; G6PD; |
| | | | DNA synthesis and cell | GLA; HN1; HSPA4; |
| | | | cycle progression | KPNA2; NRAS; |
| | | | | PDCD2; PSMD1; |
| | | | | SAE1; TAF9 |
| RARRES2 | Retinoic acid receptor | 7q36.1 | Chemotactic protein | CYP2C9; GNMT; |
| | responder (tazarotene | | | LCAT; MIA3; |
| | induced) 2 | | | UGT2B7; C8A; |
| | | | | KRT19; EPCAM; |
| | | | | AKR1C1.AKR1C2; |
| | | | | SERPIN |
| RBM47 | RNA binding motif protein | 4p14 | Unknown function | ARFGEF2; |
| | 47 | | | C14orf156; DPP8; |
| | | | | ENO1; HN1; HSPA4; |
| | | | | KPNA2; NRAS; |
| | | | | NUDT9; PDCD2; |
| | | | | PSMD1; RAN; TAF9; |
| | | | | TFRC |
| REG3A | Regenerating islet-derived | 2p12 | Pancreatic secretory | AMACR; EPHA1; |
| | 3 alpha | | protein, involved in cell | GLS2; GLUL; HAL; |
| | | | proliferation, also called | LAMA3; LGR5; |
| | | | PAP | RHBG; SLPI; TBX3 |
| RHBG | Rh family, B glycoprotein | 1q21.3 | Ammonium transporter | AMACR; CXCR7; |
| | (gene/pseudogene) | | | CYP2C9; GLUL; HAL; |
| | | | | LAMA3; LGR5; |
| | | | | REG3A; SLPI; TBX3; |
| | | | | UGT2B7; |
| | | | | AKR1C1.AKR1C2 |
| SAA2 | Serum amyloid A2 | 11p15.1-p14 | Protein of the acute | AURKA; BIRC5; |
| | | | phase of inflammation | CCNB1; CRP; ESR1; |
| | | | | HAMP; NEK7; |
| | | | | STEAP3; C8A; |
| | | | | LYVE1 |
| SAE1 | SUMO1 activating enzyme | 19q13.32 | Posttranslational | ARFGEF2; AURKA; |
| | subunit 1 | | modification of proteins, | BIRC5; CCNB1; |
| | | | sumoylation | CDC20; DPP8; |
| | | | | G6PD; GLA; HN1; |
| | | | | HSPA4; KPNA2; |
| | | | | NEK7; NRAS; |
| | | | | PSMD1; RAN; TAF9; |
| | | | | TRIP13; CKS2; |
| | | | | RRM2; DLGAP5 |
| SDS | Serine dehydratase | 12q24.21 | Metabolism of serine, | CHKA; CRP; |
| | | | glycine and ammonia | GADD45B; GLS2; |
| | | | | GLUL; GNMT; HAL; |
| | | | | LGR5; RAMP3; |
| | | | | SAA2; SLPI; C8A |
| TAF9 | TAF9 RNA polymerase II, | 5q11.2-q13.1 | transcriptional activation, | ARFGEF2; CCNB1; |
| | TATA box binding protein | | gene regulation | DPP8; HSPA4; |
| | (TBP)-associated | | associated with apoptosis | KPNA2; NRAS; RAN; |
| | factor, 32 kDa | | | SAE1 |
| TFRC | Transferrin receptor (p90, | 3q26.2-qter | Cellular uptake of iron | AURKA; BIRC5; |
| | CD71) | | | CCNB1; CDC20; |
| | | | | ENO1; G6PD; HN1; |
| | | | | HSPA4; KPNA2; |
| | | | | NRAS; RAN; SAE1; |
| | | | | TRIP13; CKS2; |
| | | | | RRM2 |
| UGT2B7 | UDP | 4q13 | Regulation of estrogen | CRP; CYP2C9; |
| | glucuronosyltransferase 2 | | metabolites | FABP1; GNMT; |
| | family, polypeptide B7 | | | RARRES2; C8A; |
| | | | | AKR1C1.AKR1C2 |

In the above methods according to the invention, in order to distinguish hepatocellular/non-hepatocellular samples, benign/malignant hepatocellular samples, FNH/non-FNH benign hepatocellular samples, HCA/non-HCA benign hepatocellular samples, HNF1A mutated/non-HNF1A mutated HCA samples, inflammatory/non-inflammatory HCA samples, and β catenin mutated/non-β catenin mutated HCA samples, expression profiles comprising or consisting of specific genes, or Equivalent Expression Profiles thereof are analyzed. By “expression profile”, it is meant the expression levels of the group of genes included in the expression profile. By “comprising”, it is intended to mean that the expression profile may further comprise other genes. In contrast, by “consisting of”, it is intended to mean that no further gene is present in the expression profile analyzed. By “Equivalent Expression Profile thereof” or “EEP”, it is intended to mean the original expression profile (to which said EEP is equivalent), wherein the addition, deletion or substitution of some of the genes (preferably at most 1 or 2 genes) does not change significantly the reliability of the diagnosis, i.e. for which the values of sensitivity (Sen), specificity (Spe), positive predictive value (PPV), and negative predictive value (NPV) are not lowered by more than 10%.

Sensitivity, specificity, PPV and NPV are usual statistical parameters well-known to those skilled in the art.

Sensitivity relates to the test's ability to identify positive results and is the proportion of people who have the disease who test positive for it.

Specificity relates to the ability of the test to identify negative results and is defined as the proportion of patients who do not have the disease who will test negative for it.

Positive predictive value (PPV) is the proportion of positive test results that are true positives.

Negative predictive value (NPV) is defined as the proportion of subjects with a negative test result who are correctly diagnosed.

In a preferred embodiment, Equivalent Expression Profiles include expression profiles in which one of the genes of a selected genes combination is replaced by an equivalent gene. In the present description, a first gene (“gene A”) can be considered as equivalent to another second gene (“gene B”), when replacing “gene A” in the expression profile of by “gene B” does not significantly impact the performance of the test, i.e. the values of sensitivity (Sen), specificity (Spe), positive predictive value (PPV), and negative predictive value (NPV) are not lowered by more than 10%. This is typically the case when “gene A” is correlated to “gene B”, meaning that the expression of “gene A” is statistically correlated to the expression level of “gene B”, as determined by a measure such as Pearson's correlation coefficient. The correlation may be positive (meaning that when “gene A” is upregulated in a patient, then “gene” B is also upregulated in that same patient) or negative (meaning that when “gene A” is upregulated in a patient, then “gene B” is downregulated in that same patient). A maximum of 10 genes among the 103 genes analyzed by the inventors using quantitative PCR, which are the best correlated to each of the 55 genes necessary for complete classification, and which have an average Pearson's correlation coefficient ≧0.3 or ≦−0.3 are mentioned in Table 2 above.

By “determining an expression profile”, it is meant the measure of the expression level of a group a selected genes. The expression level of each gene may be determined in vitro either at the proteic or at the nucleic level, using any technology known in the art. For instance, at the proteic level, the in vitro measure of the expression level of a particular protein may be performed by any dosage method known by a person skilled in the art, including but not limited to ELISA or mass spectrometry analysis. These technologies are easily adapted to any liver sample. Indeed, proteins of the liver sample may be extracted using various technologies well known to those skilled in the art for ELISA or mass spectrometry in solution measure. Alternatively, the expression level of a protein in a liver sample may be analyzed using mass spectrometry directly on the tissue slice.

In a preferred embodiment of a method according to the invention, the expression profile is determined in vitro at the nucleic level. At the nucleic level, the in vitro measure of the expression level of a gene may be carried out either directly on messenger RNA (mRNA), or on retrotranscribed complementary DNA (cDNA). Any method to measure the expression level may be used, including but not limited to microarray analysis, quantitative PCR, southern analysis. In a preferred embodiment of a method according to the invention the expression profile is determined in vitro using a nucleic acid microarray, in particular an oligonucleotide microarray. In another preferred embodiment of a method according to the invention, the expression profile is determined in vitro using quantitative PCR. In any case, the expression level of any gene is preferably normalized. There are many methods for normalizing obtained expression data, depending on the technology used for measuring expression. Such methods are well known to those skilled in the art. In some embodiments, normalization may be performed in comparison to the expression level of an internal control gene, generally a household gene, including but not limited to ribosomal RNA (such as for instance 18S ribosomal RNA) or genes such as HPRT1 (hypoxanthine phosphoribosyltransferase 1), UBC (ubiquitin C), YWHAZ (tyrosine 3-monooxygenase/tryptophan 5-monooxygenase activation protein, zeta polypeptide), B2M (beta-2-microglobulin), GAPDH (glyceraldehyde-3-phosphate dehydrogenase), FPGS (folylpolyglutamate synthase), DECR1 (2,4-dienoyl CoA reductase 1, mitochondrial), PPIB (peptidylprolyl isomerase B (cyclophilin B)), ACTB (actin β), PSMB2 (proteasome (prosome, macropain) subunit, beta type, 2), GPS1 (G protein pathway suppressor 1), CANX (calnexin), NACA (nascent polypeptide-associated complex alpha subunit), TAX1BP1 (Tax1 (human T-cell leukemia virus type I) binding protein 1), and PSMD2 (proteasome (prosome, macropain) 26S subunit, non-ATPase, 2).

In the context of the present invention, “expression values” (also referred to as “expression levels”) of genes used for the prognosis include both:
non-normalized raw expression values, and
derivatives of raw expression values, which may further have been normalized no matter with method is used for normalization.
In particular, when quantitative PCR is used for measuring in vitro expression values of genes used for prognosis, derivatives of raw expression values selected from ΔCt, −ΔCt, ΔΔCt, or −ΔΔCt values may be used.
When a microarray is used for measuring in vitro expression values of genes used for prognosis, log derivatives (in particular log 2 derivatives) of raw expression values (which may further have been normalized or not) are usually used.

These technologies are also easily adapted to any liver sample. Indeed, several well-known technologies are available to those skilled in the art for extracting mRNA from a tissue sample and retrotranscribing mRNA into cDNA.

Many algorithms may be used for interpreting expression profiles in order to distinguish hepatocellular/non-hepatocellular samples, benign/malignant hepatocellular samples, FNH/non-FNH benign hepatocellular samples, HCA/non-HCA benign hepatocellular samples, HNF1A mutated/non-HNF1A mutated HCA samples, inflammatory/non-inflammatory HCA samples, and β catenin mutated/non-β catenin mutated HCA samples. Notably, appropriate algorithms include PLS (Partial Least Square) regression, Support Vector Machines (SVM), linear regression or derivatives thereof (such as the generalized linear model abbreviated as GLM, including logistic regression), Linear Discriminant Analysis (LDA, including Diagonal Linear Discriminant Analysis (DLDA)), Diagonal quadratic discriminant analysis (DQDA), Random Forests, k-NN (Nearest Neighbour) or PAM (Predictive Analysis of Microarrays) algorithms.

A group of reference samples, which is generally referred to as training data, is used to select an optimal statistical algorithm that best separates good from bad prognosis (like a decision rule). The best separation is usually the one that misclassifies as few samples as possible and that has the best chance to perform comparably well on a different dataset.

For a binary outcome such as good/bad prognosis, linear regression or a generalized linear model (abbreviated as GLM), including logistic regression, may be used.

Linear regression is based on the determination of a linear regression function, which general formula may be represented as:

f(x₁, . . . ,x_{N})=β₀+β₁x₁+ . . . +β_{N}x_{N}.

Logistic regression is based on the determination of a logistic regression function:

$$f ( z ) = \frac{{e}^{z}}{{e}^{z} + 1} = \frac{1}{1 + {e}^{- z}} ,$$

in which z is usually defined as

z=β₀+β₁x₁+ . . . +β_{N}x_{N}.

In the above linear or logistic regression functions, x₁ to x_{N} are the expression values (or derivatives thereof such as ΔCt, −ΔCt, ΔΔCt, or −ΔΔCt for quantitative PCR or logged values for microarray) of the N genes in the signature, β₀ is the intercept, and β₁ to β_{N} are the regression coefficients.

The values of the intercept and of the regression coefficients are determined based on a group of reference samples (“training data”). The value of the linear or logistic regression function then defines the probability that a test expression profile has a good or bad prognosis (when defining the linear or logistic regression function based on training data, the user decides if the probability is a probability of good or bad prognosis). A test expression profile is then classified as having a good or bad prognosis depending if the probability that it has good or bad prognosis is inferior or superior to a particular threshold value, which is also determined based on training data. Sometimes, two threshold values are used, defining an undetermined area. Other types of generalized linear models than logistic regression may also be used.

Alternative methods such as nearest neighbour (abbreviated as k-NN) are also commonly used for a new sample, based on whether the sample is closer to the group of good prognosis or to the group of bad prognosis. The notion of “closer” is based on a choice of distance (metric, such as but not limited to Euclidian distance) in the n-dimension space defined by a signature consisting of N genes useful for prognosis (thus excluding potential housekeeping genes used for normalization purpose). The distances between a test expression profile and all reference good or bad prognosis expression profiles are calculated and the sample is classified by analysis of the k closest reference samples (k being an positive integer of at least 1 and most commonly 3 or 5), a rule of classification being pre-established depending of the number of good or bad prognosis reference expression profiles among the k closest reference expression profiles. For instance, when k is 1, a test expression profile is classified as good prognosis if the closest reference expression profile is a good prognosis expression profile, and as bad prognosis if the closest reference expression profile is a bad prognosis expression profile. When k is 2, a test expression profile is classified as responding if the two closest reference expression profiles are good prognosis expression profiles, as non-responding if the two closest reference expression profiles are bad prognosis expression profiles, and undetermined if the two closest reference expression profiles include a good prognosis and a bad prognosis reference expression profile. When k is 3, a test expression profile is classified as good prognosis if at least two of the three closest reference expression profiles are good prognosis expression profiles, and as bad prognosis if at least two of the three closest reference expression profiles are bad prognosis expression profiles. More generally, when k is p, a test expression profile is classified as good prognosis if more than half of the p closest reference expression profiles are good prognosis expression profiles, and as bad prognosis if more than half of the p closest reference expression profiles are bad prognosis expression profiles. If the numbers of good prognosis and bad prognosis reference expression profiles are equal, then the test expression profile is classified as undetermined.

Other methodologies from the field of statistics, mathematics or engineering exist, for example but not limited to decision trees, Support Vector Machines (SVM), Neural Networks and Linear Discriminant Analyses (LDA). These approaches are well known to people skilled in the art.

In summary, an algorithm (which may be selected from linear regression or derivatives thereof such as generalized linear models (GLM, including logistic regression), nearest neighbour (k-NN), decision trees, support vector machines (SVM), neural networks, linear discriminant analyses (LDA), Random forests, or Predictive Analysis of Microarrays (PAM) is calibrated based on a group of reference samples (preferably including several good prognosis reference expression profiles and several bad prognosis reference expression profiles) and then applied to the test sample. In simple terms, a patient will be classified as good prognosis (or bad prognosis) based on how all the genes in the signature compare to all the genes from a reference profile that was developed from a group of good prognosis (training data).

The notion of whether individual genes of the expression profile are increased or decreased in a good prognosis versus a bad prognosis sample is of scientific interest. For each individual gene, the gene expression levels in the good prognosis group can be compared to the bad prognosis group by the use of Student's t-test or equivalent methods. However, such binary comparisons are generally not used for prognosis when a signature comprises several distinct genes.

In a preferred embodiment, algorithm(s) used for interpreting any expression profile described herein as useful for distinguishing the above mentioned samples are selected from:
a) Prediction Analysis of Microarrays (PAM):

PAM(sample X)=Arg max(θ_{Yes}(sample X);θ_{No}(sample X))
wherein

$${θ}_{Yes} ( sample X ) = ( {\sum }_{i = 1}^{N} \frac{( {x}_{i} - {π}_{i} )}{{γ}_{i}} \times {π}_{Yes , i} ) - {K}_{Yes}$$

$${θ}_{No} ( sample X ) = ( {\sum }_{i = 1}^{N} \frac{( {x}_{i} - {π}_{i} )}{{γ}_{i}} \times {π}_{No , i} ) - {K}_{No}$$

wherein:
   xᵢ, 1≦i≦N, represent the in vitro measured values of N variables derived from the expression levels of genes of the expression profile, and
   πᵢ, γᵢ, π_{Yes,i}, π_{No,i}, 1≦i≦N, K_{Yes} and K_{No} are fixed parameters calibrated with at least one reference sample;
b) Diagonal Linear Discriminant Analysis (DLDA):

DLDA(sample X)=Arg min(Δ_{Yes}(sample X);Δ_{No}(sample X))
wherein

$${Δ}_{Yes} ( sample X ) = {\sum }_{i = 1}^{N} \frac{{( {x}_{i} - {μ}_{yes , i} )}^{2}}{{υ}_{i}}$$

$${Δ}_{No} ( sample X ) = {\sum }_{i = 1}^{N} \frac{{( {x}_{i} - {μ}_{No , i} )}^{2}}{{υ}_{i}}$$

wherein:
   xᵢ, 1≦i≦N, represent the in vitro measured values of N variables derived from the expression levels of genes of the expression profile, and
   υᵢ, μ_{Yes,i}, and μ_{No,i}, 1≦i≦N, are fixed parameters calibrated with at least one reference sample;
c) Diagonal quadratic discriminant analysis (DQDA):

DQDA(sample X)=Arg min(∇_{Yes}(sample X);∇_{No}(sample X))
wherein

$${▽}_{Yes} ( sample X ) = ( {\sum }_{i = 1}^{N} \frac{{( {x}_{i} - {μ}_{Yes , i} )}^{2}}{{v}_{Yes , i}} ) + {C}_{Yes}$$

$${▽}_{No} ( sample X ) = ( {\sum }_{i = 1}^{N} \frac{{( {x}_{i} - {μ}_{No , i} )}^{2}}{{v}_{No , i}} ) + {C}_{No}$$

wherein:
   xᵢ, 1≦i≦N, represent the in vitro measured values of N variables derived from the expression levels of genes of the expression profile, and
   υ_{Yes,i}, υ_{No,i}, μ_{Yes,i}, μ_{No,i}, 1≦i≦N, are fixed parameters calibrated with at least one reference sample, and

$${C}_{Yes} = ( {\sum }_{i = 1}^{N} log ( {v}_{Yes , i} ) )$$

$${C}_{No} = ( {\sum }_{i = 1}^{N} log ( {v}_{No , i} ) ) ;$$

d) or any combination thereof.

For the purpose of interpreting expression profiles in order to distinguish hepatocellular/non-hepatocellular samples, benign/malignant hepatocellular samples, FNH/non-FNH benign hepatocellular samples, HCA/non-HCA benign hepatocellular samples, HNF1A mutated/non-HNF1A mutated HCA samples, inflammatory/non-inflammatory HCA samples, and β catenin mutated/non-β catenin mutated HCA samples, a particularly advantageous algorithm is:

Diagnosis(sample X)=majority rule(PAM(sample X),DLDA(sample X),DQDA(sample X))

In a preferred embodiment, for the purpose of interpreting expression profiles in order to distinguish hepatocellular/non-hepatocellular samples, benign/malignant hepatocellular samples, FNH/non-FNH benign hepatocellular samples, HCA/non-HCA benign hepatocellular samples, HNF1A mutated/non-HNF1A mutated HCA samples, inflammatory/non-inflammatory HCA samples, and β catenin mutated/non-β catenin mutated HCA samples, the expression profile(s) is(are) determined using quantitative PCR and the variables and parameters of PAM, DLDA and DQDA algorithms are the following:
a) For determining if a liver sample is or not a hepatocellular sample:
   6 variables x₁ to x₆ are used as follows:

| | |
|---|---|
| x₁ | (−ΔΔCt TFRC expression level) − (−ΔΔCt C8A expression level) |
| x₂ | (−ΔΔCt AFP expression level) + (−ΔΔCt GNMT expression level) |
| x₃ | (−ΔΔCt HAL expression level) − (−ΔΔCt EPCAM expression level) |
| x₄ | (−ΔΔCt CYP3A7 expression level) − (−ΔΔCt EPCAM expression |
| | level) |
| x₅ | (−ΔΔCt FABP1 expression level) − (−ΔΔCt EPCAM expression |
| | level) |
| x₆ | (−ΔΔCt EPCAM expression level) − (−ΔΔCt HNF4A expression |
| | level) |

PAM parameters are the following:

| xᵢ | π_{No, i} | π_{Yes, i} | πᵢ | γᵢ | K_{No} | K_{Yes} |
|---|---|---|---|---|---|---|
| x₁ | 1.342931 | −0.09325912 | 2.006058 | 7.153821 | 8.151418 | 0.0932632 |
| x₂ | −1.551583 | 0.10774885 | −4.1733248 | 9.685958 | | |
| x₃ | −1.23594 | 0.08582914 | −0.9310016 | 10.17258 | | |
| x₄ | −1.524252 | 0.10585085 | 2.8897574 | 10.391148 | | |
| x₅ | −1.261254 | 0.08758709 | −1.0531553 | 10.049158 | | |
| x₆ | 1.087001 | −0.07548619 | −1.4702021 | 9.901341 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | 11.613149 | 1.3388989 | 11.690171 | 4.251989 | 4.692407 |
| x₂ | −19.201897 | −3.12967394 | 12.73627 | 22.662048 | 22.074337 |
| x₃ | −13.503695 | −0.05789783 | 17.965523 | 27.445047 | 26.883759 |
| x₄ | −12.948974 | 3.98966931 | 6.765985 | 30.609874 | 29.198065 |
| x₅ | −13.727697 | −0.17297876 | 17.267584 | 26.144739 | 25.619118 |
| x₆ | 9.292567 | −2.21761661 | 1.913791 | 25.543753 | 24.14461 |

b) For determining if a hepatocellular sample is or not a HCC sample:
   6 variables x₁ to x₆ are used as follows:

| | |
|---|---|
| x₁ | (−ΔΔCt CAP2 expression level) − (−ΔΔCt LCAT expression level) |
| x₂ | (−ΔΔCt ANGPT2 expression level) + (−ΔΔCt AURKA expression |
| | level) |
| x₃ | (−ΔΔCt CDC20 expression level) + (−ΔΔCt DHRS2 expression level) |
| x₄ | (−ΔΔCt ANGPT2 expression level) − (−ΔΔCt LYVE1 expression |
| | level) |
| x₅ | (−ΔΔCt ADM expression level) − (−ΔΔCt CDC20 expression level) |
| x₆ | Max (−ΔΔCt AFP expression level; −ΔΔCt CAP2 expression level) |

PAM parameters are the following:

| xᵢ | π_{No, i} | π_{Yes, i} | πᵢ | γᵢ | K_{No} | K_{Yes} |
|---|---|---|---|---|---|---|
| x₁ | −0.16268042 | 0.08134021 | 5.787048 | 4.542418 | 1.272916 | 0.449041 |
| x₂ | −0.22453753 | 0.11226876 | 3.035909 | 3.975872 | | |
| x₃ | −0.42378458 | 0.21189229 | 3.937962 | 6.248688 | | |
| x₄ | −0.2592874 | 0.1296437 | 4.151425 | 3.70769 | | |
| x₅ | 0.15685585 | −0.07842792 | −4.403932 | 3.840179 | | |
| x₆ | −0.01726311 | 0.00863156 | 3.696066 | 4.123495 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | 2.678847 | 7.341149 | 2.2201 | 8.37556 | 6.33819 |
| x₂ | 0.06943705 | 4.519144 | 3.255149 | 4.0793 | 3.806517 |
| x₃ | −1.96933307 | 6.891609 | 25.818236 | 13.894186 | 17.840878 |
| x₄ | 1.25620635 | 5.599034 | 1.863177 | 3.311281 | 2.831979 |
| x₅ | −1.79861246 | −5.706591 | 2.246134 | 3.814584 | 3.295449 |
| x₆ | 1.47414444 | 4.807026 | 1.020023 | 6.078697 | 4.404347 |

c) For determining if a benign hepatocellular sample is or not a FNH sample:
   12 variables x₁ to x₁₂ are used as follows:

| | |
|---|---|
| x₁ | Min (−ΔΔCt ANGPTL7 expression level; −ΔΔCt GLUL expression |
| | level) |
| x₂ | (−ΔΔCt ANGPT1 expression level) − (−ΔΔCt HMGB3 expression |
| | level) |
| x₃ | (−ΔΔCt GMNN expression level) + (−ΔΔCt RAMP3 expression |
| | level) |
| x₄ | Min (−ΔΔCt RHBG expression level; −ΔΔCt UGT2B7 expression |
| | level) |
| x₅ | Max (−ΔΔCt HAL expression level; −ΔΔCt RAMP3 expression |
| | level) |
| x₆ | Min (−ΔΔCt LGR5 expression level; −ΔΔCt UGT2B7 expression |
| | level) |
| x₇ | (−ΔΔCt RAMP3 expression level) + (−ΔΔCt UGT2B7 expression |
| | level) |
| x₈ | (−ΔΔCt RAMP3 expression level) + (−ΔΔCt RARRES2 expression |
| | level) |
| x₉ | Max (−ΔΔCt ANGPT1 expression level; −ΔΔCt RAMP3 expression |
| | level) |
| x₁₀ | Min (−ΔΔCt ANGPT1 expression level; −ΔΔCt LGR5 expression |
| | level) |
| x₁₁ | (−ΔΔCt RAMP3 expression level) − (−ΔΔCt RBM47 expression |
| | level) |
| x₁₂ | Min (−ΔΔCt GIMAP5 expression level; −ΔΔCt UGT2B7 expression |
| | level) |

PAM parameters are the following:

| xᵢ | π_{No, i} | π_{Yes, i} | πᵢ | γᵢ | K_{No} | K_{Yes} |
|---|---|---|---|---|---|---|
| x₁ | −0.18469273 | 1.0817717 | −1.72829395 | 3.243668 | 0.2800792 | 6.1260851 |
| x₂ | −0.15724871 | 0.9210281 | 0.61243528 | 2.336453 | | |
| x₃ | −0.13637923 | 0.7987926 | 1.58326744 | 2.289755 | | |
| x₄ | −0.15358836 | 0.899589 | −3.46104209 | 3.909901 | | |
| x₅ | −0.11234999 | 0.65805 | 1.19490255 | 2.017152 | | |
| x₆ | −0.11945816 | 0.6996835 | −2.27683325 | 3.334501 | | |
| x₇ | −0.15338781 | 0.8984143 | −0.04692744 | 2.922347 | | |
| x₈ | −0.14256206 | 0.8350063 | 0.60258802 | 2.277919 | | |
| x₉ | −0.11634108 | 0.6814263 | 1.54744785 | 1.913217 | | |
| x₁₀ | −0.17351058 | 1.0162762 | −1.4122167 | 3.581967 | | |
| x₁₁ | −0.15477031 | 0.9065118 | 1.45598643 | 2.048925 | | |
| x₁₂ | −0.07438928 | 0.4357086 | −1.04952428 | 2.524675 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | −2.3273759 | 1.7806145 | 4.6402628 | 0.60826433 | 4.11435 |
| x₂ | 0.245031 | 2.76437457 | 1.4145492 | 0.20686229 | 1.2570248 |
| x₃ | 1.2709924 | 3.41230679 | 1.2978397 | 0.19883833 | 1.1544917 |
| x₄ | −4.0615574 | 0.05626186 | 8.3471726 | 0.0196296 | 7.2609714 |
| x₅ | 0.9682756 | 2.52228907 | 0.6935121 | 0.30621156 | 0.6429946 |
| x₆ | −2.6751666 | 0.05626186 | 5.1618051 | 0.0196296 | 4.4910865 |
| x₇ | −0.4951798 | 2.57855093 | 3.3012094 | 0.33314121 | 2.9140701 |
| x₈ | 0.2778432 | 2.50466495 | 1.2384457 | 0.40087507 | 1.1291973 |
| x₉ | 1.3248621 | 2.85116431 | 0.5424233 | 0.11837803 | 0.487113 |
| x₁₀ | −2.0337258 | 2.22805082 | 6.3954525 | 0.30614496 | 5.601195 |
| x₁₁ | 1.1388737 | 3.31336105 | 0.7211325 | 0.52047864 | 0.6949603 |
| x₁₂ | −1.2373331 | 0.05049854 | 1.9692555 | 0.01620956 | 1.7145104 |

d) For determining if a benign hepatocellular sample is or not a HCA sample:
   10 variables x₁ to x₁₀ are used as follows:

| | |
|---|---|
| x₁ | (−ΔΔCt AKR1B10 expression level) + (−ΔΔCt GLS2 expression |
| | level) |
| x₂ | (−ΔΔCt LCAT expression level) − (−ΔΔCt KRT19 expression level) |
| x₃ | (−ΔΔCt ESR1 expression level) + (−ΔΔCt SDS expression level) |
| x₄ | Max (−ΔΔCt MERTK expression level; −ΔΔCt LYVE1 expression |
| | level) |
| x₅ | Max (−ΔΔCt EPHA1 expression level; −ΔΔCt KRT19 expression |
| | level) |
| x₆ | (−ΔΔCt CCL5 expression level) + (−ΔΔCt GLS2 expression level) |
| x₇ | (−ΔΔCt HAL expression level) − (−ΔΔCt MERTK expression level) |
| x₈ | (−ΔΔCt CYP2C9 expression level) − (−ΔΔCt MERTK expression |
| | level) |
| x₉ | (−ΔΔCt CCL5 expression level) + (−ΔΔCt KRT19 expression level) |
| x₁₀ | Min (−ΔΔCt CYP3A7 expression level; −ΔΔCt EPHA1 expression |
| | level) |

PAM parameters are the following:

| xᵢ | π_{No,i} | π_{Yes,i} | πᵢ | γᵢ | K_{No} | K_{Yes} |
|---|---|---|---|---|---|---|
| x₁ | 1.1300586 | −0.52467006 | −0.96573089 | 5.405409 | 3.0655113 | 0.7945744 |
| x₂ | −0.6257754 | 0.29053858 | 0.10777331 | 4.174906 | | |
| x₃ | −0.583684 | 0.27099612 | 1.53413349 | 3.92968 | | |
| x₄ | −0.2101061 | 0.09754928 | 0.01545178 | 2.53848 | | |
| x₅ | 0.4031816 | −0.18719147 | 0.76400666 | 2.906802 | | |
| x₆ | 0.6342941 | −0.29449369 | −1.82990856 | 4.756332 | | |
| x₇ | 0.5211003 | −0.24193944 | −0.57174662 | 4.026102 | | |
| x₈ | 0.3773559 | −0.17520095 | −0.97286634 | 3.529012 | | |
| x₉ | 0.8070427 | −0.3746984 | −0.75070901 | 3.946451 | | |
| x₁₀ | 0.3875215 | −0.17992069 | 0.02720304 | 2.927056 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | 5.142698 | −3.8017871 | 1.9223207 | 16.202619 | 11.8086811 |
| x₂ | −2.5047803 | 1.3207446 | 4.8696186 | 4.8642148 | 4.8658775 |
| x₃ | −0.759558 | 2.5990617 | 1.5948539 | 4.8438216 | 3.8441392 |
| x₄ | −0.5178985 | 0.2630787 | 0.1157701 | 0.4169368 | 0.3242701 |
| x₅ | 1.9359758 | 0.2198781 | 0.9741474 | 0.8373057 | 0.8794108 |
| x₆ | 1.1870048 | −3.2306184 | 0.5402267 | 10.9818415 | 7.769037 |
| x₇ | 1.5262567 | −1.5458196 | 1.0506355 | 5.6452689 | 4.2315355 |
| x₈ | 0.358827 | −1.5911525 | 0.2637763 | 3.3978705 | 2.4335338 |
| x₉ | 2.4342454 | −2.2294378 | 3.9252834 | 3.9034702 | 3.910182 |
| x₁₀ | 1.1615001 | −0.4994349 | 0.507857 | 1.1000088 | 0.9178082 |

e) For determining if a HCA sample is or not a HNF1A mutated HCA sample:
   2 variables x₁ to x₆ are used as follows:

| | |
|---|---|
| x₁ | (−ΔΔCt DHRS2 expression level) − (−ΔΔCt UGT2B7 expression |
| | level) |
| x₂ | (−ΔΔCt ANGPT2 expression level) + (−ΔΔCt FABP1 expression |
| | level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.2597076 | 1.817954 | −1.130125 | 6.501417 | 0.1803095 | 4.3715711 |
| x₂ | −0.1615805 | 1.131063 | 1.136677 | 3.83618 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | −2.8185929 | 10.68915 | 15.46252 | 14.3631833 | 15.343027 |
| x₂ | 0.5168253 | 5.47564 | 1.668767 | 0.7321017 | 1.566956 |

f) For determining if a HCA sample is or not an inflammatory HCA sample:
   4 variables x₁ to x₆ are used as follows:

| | |
|---|---|
| x₁ | (−ΔΔCt HAMP expression level) + (−ΔΔCt SAA2 expression level) |
| x₂ | (−ΔΔCt CCL5 expression level) − (−ΔΔCt NRCAM expression level) |
| x₃ | Max (−ΔΔCt EPHA1 expression level; −ΔΔCt KRT19 expression |
| | level) |
| x₄ | (−ΔΔCt ANGPT2 expression level) + (−ΔΔCt SAA2 expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.4760712 | 0.9521423 | 4.6430007 | 6.107883 | 0.7344381 | 2.4145044 |
| x₂ | 0.434627 | −0.869254 | −0.0574931 | 5.002872 | | |
| x₃ | 0.1882468 | −0.3764937 | 1.1521703 | 3.158128 | | |
| x₄ | −0.4549338 | 0.9098677 | 4.5882009 | 4.501345 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | 1.735214 | 10.4585747 | 16.9585649 | 7.6603747 | 13.9265464 |
| x₂ | 2.11689 | −4.4062595 | 7.0569419 | 6.5761749 | 6.90017 |
| x₃ | 1.746678 | −0.0368447 | 0.7298408 | 0.3673544 | 0.6116387 |
| x₄ | 2.540387 | 8.6838292 | 4.4787841 | 4.5955546 | 4.5168614 |

g) For determining if a HCA sample is or not a β catenin mutated HCA sample:
   9 variables x₁ to x₆ are used as follows:

| | |
|---|---|
| x₁ | (−ΔΔCt AKR1B10 expression level) − (−ΔΔCt REG3A expression |
| | level) |
| x₂ | (−ΔΔCt AMACR expression level) + (−ΔΔCt HAL expression level) |
| x₃ | (−ΔΔCt CAP2 expression level) − (−ΔΔCt GLUL expression level) |
| x₄ | (−ΔΔCt HAL expression level) + (−ΔΔCt TAF9 expression level) |
| x₅ | (−ΔΔCt CAP2 expression level) − (−ΔΔCt LGR5 expression level) |
| x₆ | Min (−ΔΔCt AKR1B10 expression level; −ΔΔCt HAL expression |
| | level) |
| x₇ | (−ΔΔCt LAPTM4B expression level) + (−ΔΔCt TFRC expression |
| | level) |
| x₈ | (−ΔΔCt GIMAP5 expression level) − (−ΔΔCt HAL expression level) |
| x₉ | (−ΔΔCt HMGB3 expression level) − (−ΔΔCt IGF2BP3 expression |
| | level) |

PAM parameters are the following:

| xᵢ | π_{No,i} | π_{Yes,i} | πᵢ | γᵢ | K_{No} | K_{Yes} |
|---|---|---|---|---|---|---|
| x₁ | 0.34708654 | −1.9668237 | 1.94438201 | 7.392962 | 0.3607787 | 8.2634614 |
| x₂ | 0.21863143 | −1.2389115 | −1.04516656 | 3.127947 | | |
| x₃ | 0.18579207 | −1.0528217 | 1.22379671 | 2.663529 | | |
| x₄ | 0.24406366 | −1.3830274 | 0.05214403 | 3.244264 | | |
| x₅ | 0.15694722 | −0.8893676 | 2.7521494 | 3.869139 | | |
| x₆ | 0.21470021 | −1.2166345 | −1.47714108 | 4.260375 | | |
| x₇ | 0.11140632 | −0.6313025 | 0.81968112 | 3.203963 | | |
| x₈ | −0.22080529 | 1.25123 | 0.49103172 | 3.193991 | | |
| x₉ | 0.04764503 | −0.2699885 | 0.56180483 | 3.025541 | | |

DLDA and DQDA parameters are the same, as follows:

| xᵢ | μ_{No,i} | μ_{Yes,i} | υ_{No,i} | υ_{Yes,i} | υᵢ |
|---|---|---|---|---|---|
| x₁ | 4.5103796 | −12.5962709 | 37.671414 | 6.2381109 | 33.535453 |
| x₂ | −0.361299 | −4.920416 | 1.426277 | 8.2837077 | 2.328571 |
| x₃ | 1.7186592 | −1.5804241 | 1.203395 | 0.6218992 | 1.126882 |
| x₄ | 0.8439509 | −4.4347616 | 1.358794 | 11.5298442 | 2.69709 |
| x₅ | 3.3594 | −0.6889375 | 5.646265 | 1.7986761 | 5.140003 |
| x₆ | −0.5624378 | −6.6604599 | 6.819184 | 8.7029888 | 7.067053 |
| x₇ | 1.1766229 | −1.2029889 | 2.912529 | 0.2815287 | 2.566345 |
| x₈ | −0.2142184 | 4.4874493 | 1.580383 | 8.8316336 | 2.534495 |
| x₉ | 0.7059568 | −0.2550566 | 2.287403 | 0.3047094 | 2.026522 |

The present invention also relates to a kit comprising reagents for the determination of an expression profile comprising at most 65 distinct genes, wherein said expression profile is selected from:
An expression profile comprising or consisting of the following 38 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9, and optionally one or more internal control gene, or an Equivalent Expression Profile thereof;
An expression profile comprising or consisting of the following 46 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, and IGF2BP3, and optionally one or more internal control gene, or an Equivalent Expression Profile thereof;
An expression profile comprising or consisting of the following 49 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, RAB1A, REG3A, NRAS, PIR, LAMA3, G0S2, HN1, PAK2, CDH2, HAMP, and SAE1, and optionally one or more internal control gene, or an Equivalent Expression Profile thereof; or
An expression profile comprising or consisting of the following 55 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, IGF2BP3, RAB1A, NRAS, PIR, LAMA3, G0S2, HN1, PAK2, CDH2, and SAE1, and optionally one or more internal control gene, or an Equivalent Expression Profile thereof.

The kit according to the invention is preferably dedicated to the determination or one of the above mentioned expression profiles, and thus comprises reagents for the determination of an expression profile comprising at most 65 distinct genes, knowing that the expression profile with the highest number of genes of interest comprises 55 genes, and optionally one or more internal control gene. When the expression profile comprises less than 55 genes of interest, the kit preferably comprises reagents for the determination of an expression profile comprising the number of genes of interest and no more than about 10 additional genes, which may include internal control genes and/or a few additional genes. Such additional genes might correspond to a further expression profile that might be used for instance for prognosis of the disease if the sample is determined as a HCC sample.

For instance, when the expression profile comprises 49 genes of interest and optionally one or more internal control gene, the kit preferably comprises reagents for the determination of an expression profile comprising at most 59 distinct genes. When the expression profile comprises 46 genes of interest and optionally one or more internal control gene, the kit preferably comprises reagents for the determination of an expression profile comprising at most 56 distinct genes. When the expression profile comprises 38 genes of interest and optionally one or more internal control gene, the kit preferably comprises reagents for the determination of an expression profile comprising at most 48 distinct genes.

In all the above mentioned embodiments of a kit comprising reagents for the determination of an expression profile comprising at most N distinct genes, N being an integer as mentioned above, reagents comprised in the kit do not permit determination of an expression profile comprising more than N genes. In particular, such a kit according to the invention excludes pangenomic microarrays permitting determination of expression profiles of thousands of genes.

Reagents for the determination of an expression profile comprising N genes may include any reagents permitting to specifically quantify the expression levels of the genes included in said expression profile. For instance, when the expression profile is determined at the proteic level, then such reagents may include antibodies specific for each of the genes included in the expression profile. Preferably, the expression is determined at the nucleic level. In this case, reagents in the kit of the invention may notably include primers pairs (forward and reverse primers) and/or probes specific for each of the genes included in the expression profile (useful notably for quantitative PCR determination of the expression profile) or a nucleic acid microarray, in particular an oligonucleotide microarray. In the latter case, the nucleic acid microarray is a dedicated nucleic acid microarray, comprising probes for the detection of a maximum number of genes, as defined in the previous paragraph. In other words, the nucleic acid microarray does not permit determination of an expression profile comprising more than the maximum number of genes comprised in the expression profile.

As indicated in introduction, the classification method according to the invention is important for clinicians because it will permit them, based on a unique and simple test, to know precisely of which type of liver disease a subject is suffering, and thus to adapt the treatment to the precise diagnosis.

The invention thus also relates to an IGFR1 inhibitor, an Akt/mTor inhibitor, a proteasome inhibitor and/or a wnt inhibitor, for use in the treatment of HCC in a subject that has been diagnosed as suffering from HCC based on a liver sample that has been classified as a HCC sample by the classification method of the invention. The invention also relates to the use of an IGFR1 inhibitor, an Akt/mTor inhibitor, aproteasome inhibitor and/or a wnt inhibitor for the preparation of a medicament intended for the treatment of HCC in a subject that has been diagnosed as suffering from HCC based on a liver sample that has been classified as a HCC sample by the classification method of the invention. If the liver sample of said subject has been further classified as subgroup G1, then a IGFR1 inhibitor or an Akt/mTor inhibitor is preferred. If the liver sample of said subject has been further classified as subgroup G2, then an Akt/mTor inhibitor is preferred. If the liver sample of said subject has been further classified as subgroup G3, then a proteasome inhibitor is preferred. If the liver sample of said subject has been further classified as subgroup G5 or G6, then a wnt inhibitor is preferred. However, current WNT inhibitors have toxicity problems, and there is still a need for more efficient and safer WNT inhibitors.

The invention also relates to a method for treating a liver disease in a subject in need thereof, comprising:
a) Classifying a liver sample of said subject as a non-hepatocellular sample, a hepatocellular carcinoma (HCC) sample, a focal nodule dysplasia (FNH) sample, a hepatocellular adenoma (HCA) sample or another benign liver sample with the classification method according to the invention;
b) If said sample is a non-hepatocellular sample, then identifying the precise histological subtype of sample and administering to said subject a treatment according to the histological subtype identified;
c) If said sample is a HCC sample, then performing surgical resection with or without adjuvant treatment;
d) If said sample is a FNH sample, then no therapeutic action is performed;
e) If said sample is a HCA sample, then only following up the subject or performing surgical resection, depending on the HCA subgroup;
f) If said sample is another benign hepatocellular sample, then no therapeutic action is performed.

The method of treatment of the invention may further comprise, if said liver sample is a HCC sample:
i. classifying said HCC sample into one of subgroups G1 to G6 as described above; and
ii. if said HCC sample is classified in G1 subgroup, then administering an efficient amount of an IGFR1 inhibitor or of an Akt/mTor inhibitor to said patient;
iii. if said HCC sample is classified in G1-G2 subgroup, administering an efficient amount of an hen Akt/mTor inhibitor to said patient;
iv. if said HCC sample is classified in G3 subgroup, then administering an efficient amount of a proteasome inhibitor to said patient;
v. if said HCC sample is classified in G5-G6 subgroup, then administering an efficient amount of a wnt inhibitor to said patient.

The method of treatment of the invention may further comprise, if said liver sample is a HCC sample:
i. Prognosing global survival and/or survival without relapse; and
ii. if said HCC sample is given a good prognosis, then no adjuvant treatment is performed;
iii. if said HCC sample is given a bad prognosis, then administering to said subject an adjuvant treatment, such as cytotoxic chemotherapy and/or targeted therapy.

According to the invention, a “prognosis” of HCC evolution means a prediction of the future evolution of a particular HCC tumor relative to the patient suffering of this particular HCC tumor. The method according to the invention allows simultaneously for both a global survival prognosis and a survival without relapse prognosis.

By “global survival prognosis” is meant prognosis of survival, with or without relapse. As stated before, the main current treatment against HCC is tumor surgical resection. As a result, a “bad global survival prognosis” is defined as the occurrence of death within the 3 years after liver resection, whereas a “good global survival prognosis” is defined as the lack of death during the 5 post-operative years.

By “survival without relapse prognosis” is meant prognosis of survival in the absence of any relapse. A “bad survival without relapse prognosis” is defined as the presence of tumor-relapse within the two years after liver resection, whereas a “good survival without relapse prognosis” is defined as the lack of relapse during the 4 post-operative years.

Such prognosis of global survival and/or survival without relapse may be performed using any suitable method. Examples of such methods are notably described in WO2007/063118A1.

Adjuvants treatments are administered in case of bad prognosis. Said adjuvant treatment may be selected from:
a) cytotoxic chemotherapy, i.e. therapy with any suitable chemical agent useful for killing cancer cells. Cytotoxic chemotherapeutic agents currently used as adjuvant treatment of HCC and preferred in the present invention are doxorubicin, gemcitabine, oxaliplatine, and combinations thereof. Doxorubicin or association of gemcitabine and oxaliplatine are particularly preferred.
b) targeted therapy, i.e. therapy with any suitable agent that selectively inhibits enzymes of a signaling pathway involved in HCC malignant transformation. Currently, Sorafenib, a small molecular inhibitor of several Tyrosine protein kinases (VEGFR and PDGFR) and Raf kinases (more avidly C-Raf than B-Raf), is approved for the adjuvant treatment of HCC is is preferred in the present invention. Sorafenib is a bi-aryl urea of formula:

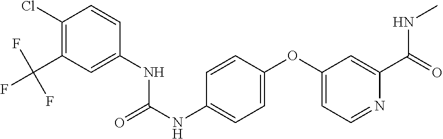 [SMILES: CNC(=O)c1cc(Oc2ccc(NC(=O)Nc3ccc(Cl)c(C(F)(F)F)c3)cc2)ccn1]

The method of treatment of the invention may also further comprise, if said liver sample is a HCA sample:
i. classifying said HCA sample into one of subgroups HNF1A mutated HCA, inflammatory HCA, β catenin mutated HCA or other HCA as described above; and
ii. if said HCA sample is classified as a HNF1A mutated HCA sample, then only following up said subject if HCA<5 cm, or performing surgical resection if HCA>5 cm;
iii. if said HCA sample is classified as an inflammatory HCA sample, then only following up said subject if HCA<5 cm, or performing surgical resection if HCA>5 cm;
iv. if said HCA sample is classified as a β catenin mutated HCA sample, then performing surgical resection whatever the HCA size.

The present invention also relates to systems (and computer readable medium for causing computer systems) to perform a method of classification of liver samples according to the invention.

In an embodiment, the invention relates to a system 1 for classifying a liver sample comprising:
a) a determination module 2 configured to receive a liver sample and to determine expression level information concerning:
   An expression profile comprising or consisting of the following 38 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9, and optionally one or more internal control genes, or an Equivalent Expression Profile thereof;
   An expression profile comprising or consisting of the following 46 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, and IGF2BP3, and optionally one or more internal control genes, or an Equivalent Expression Profile thereof;
   An expression profile comprising or consisting of the following 49 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, RAB1A, REG3A, NRAS, PIR, LAMA3, G0S2, HN1, PAK2, CDH2, HAMP, and SAE1, and optionally one or more internal control genes, or an Equivalent Expression Profile thereof; or
   An expression profile comprising or consisting of the following 55 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, IGF2BP3, RAB1A, NRAS, PIR, LAMA3, G0S2, HN1, PAK2, CDH2, and SAE1, and optionally one or more internal control genes, or an Equivalent Expression Profile thereof.
b) a storage device 3 configured to store the expression level information from the determination module;
c) a comparison module 4, adapted to compare the expression level information stored on the storage device with reference data, and to provide a comparison result, wherein the comparison result is indicative of the type of liver sample; and
d) a display module 5 for displaying a content 6 based in part on the classification result for the user, wherein the content is a signal indicative of the type of liver sample.

In another embodiment, the invention relates to a computer readable medium 7 having computer readable instructions recorded thereon to define software modules for implementing on a computer steps of a classification method according to the invention relating to interpretation of expression profiles data. Preferably, said software modules comprising:
a) an entry module 8, which permits expression level information to be entered by a user and to be stored (at least temporarily) for further comparison, wherein said expression level information relates to:
   An expression profile comprising or consisting of the following 38 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9, and optionally one or more internal control genes, or an Equivalent Expression Profile thereof;
   An expression profile comprising or consisting of the following 46 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, and IGF2BP3, and optionally one or more internal control genes, or an Equivalent Expression Profile thereof;
   An expression profile comprising or consisting of the following 49 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, RAB1A, REG3A, NRAS, PIR, LAMA3, G0S2, HN1, PAK2, CDH2, HAMP, and SAE1, and optionally one or more internal control genes, or an Equivalent Expression Profile thereof; or
   An expression profile comprising or consisting of the following 55 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, IGF2BP3, RAB1A, NRAS, PIR, LAMA3, G0S2, HN1, PAK2, CDH2, and SAE1, and optionally one or more internal control genes, or an Equivalent Expression Profile thereof;
b) a comparison module 4, adapted to compare the expression level information entered by the user with reference data and to provide a comparison result, wherein the comparison result is indicative of the type of liver sample; and
c) a display module 5, for displaying a content 6 based in part on the comparison result for the user, wherein the content is a signal indicative of the type of liver sample.

Embodiments of the invention relating to systems and computer-readable media have been described through functional modules, which are defined by computer executable instructions recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable medium can be any available tangible media that can be accessed by a computer. Computer readable medium includes volatile and nonvolatile, removable and non-removable tangible media implemented in any method or technology for storage of information such as computer readable instructions, data structures, program modules or other data. Computer readable medium includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. Computer-readable data embodied on one or more computer-readable media, may define instructions, for example, as part of one or more programs, that, as a result of being executed by a computer, instruct the computer to perform one or more of the functions described herein (e.g., in relation to system 1, or computer readable medium 7), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either system 1, or computer readable medium 6 described herein, may be distributed across one or more of such components, and may be in transition there between.

The computer-readable media may be transportable such that the instructions stored thereon can be loaded onto any computer resource to implement the aspects of the present invention discussed herein. In addition, it should be appreciated that the instructions stored on the computer readable media, or the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997, ref 38); Salzberg, Searles, Kasif, (Ed.), Computational Methods in Molecular Biology, (Elsevier, Amsterdam, 1998, ref 39); Rashidi and Buehler, Bioinformatics Basics: Application in Biological Science and Medicine (CRC Press, London, 2000, ref 40) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module 2, a storage device 3, a comparison module 4 and a display module 5. The functional modules can be executed on one, or multiple, computers, or by using one, or multiple, computer networks. The determination module 2 has computer executable instructions to provide expression level information in computer readable form.

As used herein, “expression level information” refers to information about expression level of any nucleotide (RNA or DNA) and/or amino acid sequences, either full-length or partial. In a preferred embodiment, it refers to the level of expression of mRNA or cDNA, measured by various technologies. The information may be qualitative (presence or absence of a transcript) or quantitative. Preferably it is quantitative. Methods for determining expression level information, i.e. determination modules 2, include systems for protein and DNA/RNA analysis, and in particular those described above for determination of expression profiles at the nucleic or protein level.

The expression level information determined in the determination module can be read by the storage device 3. As used herein the “storage device” 3 is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices 3 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 3 is adapted or configured for having recorded thereon expression level information. Such information may be provided in digital form that can be transmitted and read electronically, e.g., via the Internet, on diskette, via USB (universal serial bus) or via any other suitable mode of communication including wireless communication between devices.

As used herein, “stored” refers to a process for encoding information on the storage device 3. Those skilled in the art can readily adopt any of the presently known methods for recording information on known media to generate manufactures comprising the expression level information.

A variety of software programs and formats can be used to store the expression level information on the storage device. Any number of data processor structuring formats (e.g., text file, spreadsheets or database) can be employed to obtain or create a medium having recorded thereon the expression level information.

By providing expression level information in computer-readable form, one can use the expression level information in readable form in the comparison module 4 to compare a specific expression profile with the reference data within the storage device 3. The comparison may notably be done using the various algorithms described above. The comparison made in computer-readable form provides a computer readable comparison result which can be processed by a variety of means. Content based on the comparison result can be retrieved from the comparison module 4 and displayed by the display module 5 to indicate the type of liver sample.

Preferably, reference data are expression level profiles that are indicative of all types of liver samples that may be found by a classification method according to the invention. The “comparison module” 4 can use a variety of available software programs and formats for the comparison operative to compare expression level information determined in the determination module 2 to reference data, either directly, or indirectly using any software providing statistical classification algorithms such as those already described above.

The comparison module 4, or any other module of the invention, may include an operating system (e.g., Windows, Linux, Mac OS or UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module 4 provides computer readable comparison result that can be processed in computer readable form by predefined criteria, or criteria defined by a user, to provide a content 6 based in part on the comparison result that may be stored and output as requested by a user using a display module 5. The display module 5 enables display of a content 6 based in part on the comparison result for the user, wherein the content is a signal indicative of the type of liver sample. Such signal can be, for example, a display of content indicative of the type of liver sample on a computer monitor, a printed page or printed report of content indicating the type of liver sample from a printer, or a light or sound indicative of the type of liver sample.

The display module 5 can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or from ARM Holdings, or any other type of processor, visual display devices such as flat panel displays, cathode ray tubes and the like, as well as computer printers of various types or integrated devices such as laptops or tablets, in particular iPads.

In one embodiment, a World Wide Web browser is used for providing a user interface for display of the content 6 based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information.

In one embodiment, the display module 5 displays the comparison result and whether the comparison result is indicative of the type of liver sample.

In one embodiment, the content 6 based on the comparison result that is displayed is a signal (e.g. positive or negative signal) indicative of the type of liver sample, thus only a positive or negative indication may be displayed.

The present invention therefore provides for systems 1 (and computer readable media 7 for causing computer systems) to perform methods of classifying liver samples, based on expression profiles information.

System 1, and computer readable medium 7, are merely illustrative embodiments of the invention for performing methods of classification of liver sample based on expression profiles, and are not intended to limit the scope of the invention. Variations of system 1, and computer readable medium 7, are possible and are intended to fall within the scope of the invention.

The modules of the system 1 or used in the computer readable medium, may assume numerous configurations. For example, function may be provided on a single machine or distributed over multiple machines.

Having generally described this invention, a further understanding of characteristics and advantages of the invention can be obtained by reference to certain specific examples and figures which are provided herein for purposes of illustration only and are not intended to be limiting unless otherwise specified.

### DESCRIPTION OF THE FIGURES

FIG. 1: a 55 genes molecular algorithm for the classification and diagnosis of hepatocellular tumors. Sensitivity (sen), specificity (spe), negative predictive value (PNV), positive predictive value (PPV) and accuracy (acc) were detailed underneath each subset of tumors. Genes in each branch of the algorithm were resumed inside the grey boxes.

### EXAMPLES

### Example 1

### Identification of Molecular Signatures Permitting to Classify a Liver Sample Among Various Types of Liver Disease

### Patients and Methods

### Patients and Tissue Samples

Liver samples were systematically frozen following liver resection for tumor in two French University hospitals, in Bordeaux (from 1998 to 2007) and Créteil (From 2003 to 2007). A total of 550 samples were included in this work and the study was approved by the local IRB committee (CCPRB Paris Saint Louis, 1997 and 2004) and all patients gave their informed consent according to French law. Were excluded: (1) tumors with necrosis>80%, (2) tumors with RNA of poor quality or of insufficient amount, (3) HCC with non-curative resection: R1 or R2 resection or extra hepatic metastasis at the time of the surgery, (4) HCC treated by liver transplantation.

Accordingly, the following samples were included:
40 non-hepatocellular tumors, comprising intra-hepatic cholangiocarcinoma (n=19), metastasis of colorectal (n=14) and neuroendocrine (n=2) carcinoma, angiolipoma (n=3), leiomyoma (n=1) and angioma (n=1),
324 HCC,
156 benign hepatocellular tumors, including focal nodular hyperplasia (FNH, n=25), hepatocellular adenoma (HCA, n=111), regenerative macronodule (with dysplasia, n=15, or without, n=5), and
30 non-tumor samples, including cirrhosis (n=23 associated to HCV n=10, HBV n=3, alcohol n=7, NASH n=1, primary biliary cirrhosis n=1, alpha-1 antitrypsin deficiency n=1) and 7 normal liver tissues.

Molecular subtypes of HCA (β-catenin activated n=23, HNF1A inactivated n=26, inflammatory n=68 and unclassified n=8) were determined according to the previous molecular classification described in Zucman Rossi J, et al. Hepatology 2006, using gene mutation and immunohistochemistry staining. 14 (12.6%) HCA exhibited both an inflammatory phenotype and activating mutations of β-catenin.

Tumor and non-tumor liver samples were frozen immediately after surgery and conserved at −80° C. Tissue samples from the frozen counterpart were also fixed in 10% formaldehyde, paraffin-embedded and stained with Hematoxylin and Eosin and Masson's trichrome. The diagnosis of HCA, HCC, FNH, macroregenerative nodule and all non-hepatocellular tumors was based on established histological criteria (International working party Hepatology 1995, international consensus group Hepatology 2009). All tumors were assessed independently by 2 expert pathologists (JC and PBS) without knowledge of patient's outcome and initial diagnosis. In case of disagreement regarding the subtype diagnosis of hepatocellular tumors or regarding the pathological features of HCC included in prognosis analysis, sections were re-examined and a consensus was reached and used for the study. In the case of multitumors, the largest nodule available was analysed in our prognostic study.

### Selection of Genes for Further Analysis by Quantitative PCR

We selected 103 genes for the quantitative RT-PCR analysis. Using Affymetrix HG133A gene chip TM microarray hybridizations performed on the same platform, the mRNA expression of 82 liver samples including 57 HCC (E-TABM-36), 5 HNF1A inactivated adenomas (GSE7473), 7 inflammatory adenomas (GSE11819), 4 focal nodular hyperplasia (GSE9536) 9 non-tumor liver samples including cirrhosis and normal livers (E-TABM-36 and GSE7473) was analyzed. Genes differentially expressed in specific subgroups of tumors were selected according to 3 criteria for inclusion:
(1) 38 genes were selected from previous microarray data obtained by the inventors and described in boyault et al and rebouissou J B C Rebouissou Nature and rebouissou J Hepatol: RAB1A, REG3A, NRAS, RAMP3, MERTK, PIR, EPHA1, LAMA3, G0S2, HN1, PAK2, AFP, CYP2C9, CDH2, HAMP, SAE1, NTS, HAL, SDS, cmkOR1/CXCR7, ID2, GADD45B, CDT6, UGT2B7, LFABP, GLUL, LGR5/GPR49, TBX3, RHBG, SLPI, AMACR, SAA2, CRP, MME, DHRS2, SLC16A1, GLS2, and GNMT;
(2) 9 genes were previously described in the literature (Odom D T, et al. 2004; Paradis V, et al. 2003; Rebouissou S, et al. 2008; Llovet J, et al. 2006; Capurro M, et al. 2003; Chuma M, et al. 2003; Tsunedomi 2005; Kondoh N 1999): HNF1A, HNF4A, SERPIN, ANGPT1, ANGPT2, XLKD1-LYVE1, GPC3, HSP70/HSPA1A, and CYP3A7; and
(3) 13 genes were selected from new analysis of previous microarray data of the inventors: STEAP3, RRM2, GSN, CYP2C19, C8A, AKR1B10, ESR1, GMNN, CAP2, DPP8, LCAT, NEK7, LAPTM4B.

A total of 60 genes were selected for further analysis by quantitative PCR.

At this stage, the inventors also wished to provide a new tool for simple and reliable prognosis of HCC, so that further genes found or already described as associated to HCC prognosis were also included for further quantitative PCR analysis:
(1) a panel of 41 genes mostly differentially expressed (significance and fold change) between HCC patients characterized by radically different prognosis was identified by new microarray data obtained using Affymetrix microarray E-TABM-36 analysis of the pattern of expression of 44 HCC treated by curative resection: TAF9, NRCAM, PSMD1, ARFGEF2, SPP1, CDC20, NRAS, ENO1, RRAGD, CHKA, RAN, TRIP13, IMP-3/IGF2BP3, KLRB1, C14orf156, NPEPPS, PDCD2, PHB, KIAA0090, KPNA2, KIAA0268/UNQ6077/LOC440751, G6PD, STK6, TFRC, GLA, AKR1C1/AKR1C2, GIMAP5, ADM, CCNB1, TKT, ALPS, NUDT9, HLA-DQA1, NEU1, RARRES2, BIRC5, FLJ20273, HMGB3, MPPE1, CCL5, and DLG7; and
(2) 2 genes (KRT19 and EPCAM) described in the literature as related to HCC prognosis (Lee J S nat med 2006, Yamashita T gastroenterology 2008).

A total of 43 genes were selected for their association with HCC prognosis.

### Quantitative RT-PCR

RNAs extraction and quantitative RT-PCR was performed, as previously described. Expression of the 103 selected genes was analysed in duplicate in all the 550 samples using TaqMan Microfluidic card TLDA (Applied Biosystems) gene expression assays. Gene expression was normalized with the RNA ribosomal 18S, and the level of expression of the tumor sample was compared with the mean level of the corresponding gene expression in normal liver tissues, expressed as an n-fold ratio. The relative amount of RNA was calculated with the 2-delta delta CT method.

### Mutation Screening

DNA was extracted and quality was assessed. All HCA samples have been sequenced for CTNNB1 (exon 2 to 4), HNF1A (exon 1 to 10), IL6ST (exon 6 and 10), GNAS (exon 8) and STAT3 (exon 2, 5 and 20). All HCC samples have been sequenced for CTNNB1 (exon 2 to 4) and TP53 (exons 2 to 11). All mutations were confirmed by sequencing a second independent amplification product on both strands; screening for mutations in the matched non-tumor sample was performed in order to detect any germline mutations.

### Endpoints for the Diagnosis

Consensus between pathologists was considered as the gold standard for the diagnosis. We assessed sensitivity (Sen), specificity (Spe), predictive negative value (PNV), predictive positive value (PPV) and the accuracy for the diagnosis of HCC, FNH, HCA and the different subtype of HCA. Non-hepatocellular tumors, regenerative macro nodule and non-tumor liver samples (cirrhosis and normal liver) were included in order to assess the ability of the molecular algorithm to distinguish them from HCC, FNH and HCA. The study was not designed to diagnose the specific subtypes of non-hepatocellular tumors, the different subtypes of non-tumor liver samples (normal liver and cirrhosis) and of regenerative macronodules.

### Construction of the Molecular Diagnostic Algorithm

The 550 samples were divided into a global training set S1 (n=306) and a global validation set S2 (n=244). This partition was built randomly in order to provide for each variable V to be predicted (hepatocellular type, malignancy, . . . ) a training set S1_{V} (⊂S1) and a validation set S2_{V} (⊂S2) both containing approximately 50% of the samples to be analyzed for this variable and with similar proportion of “positive” cases (here all variables are binary, values being either Yes or No; “positives” cases refer to samples taking the value Yes).

103 genes were measured (−ΔΔCt measures), and four operators (addition, subtraction, min, max) were applied to all pairs of distinct genes (n=5886) to create new variables, yielding a total of 23653 variables (103 initial, 23544 created).

Given a variable V to be predicted the corresponding training set S1_{V} was randomly divided into two subsets S1_{V}.A and S1_{V}.B with equal*size and equal*proportion of “positive” cases (*:or almost equal when n is impair).

Then depending on the variable to be predicted (i.e. on the clinical implications) either a criterion giving more weight to Positive Predictive Value (focal nodular hyperplasia, HNF1A, Inflammatory, β catenin), or to Sensitivity (hepatocellular, malignancy, adenoma) was chosen. In all cases, the final criterion was obtained as 0.8 criterion₁⁴+0.2 criterion₂ (criterion₁ and criterion₂ corresponding respectively to PPV and sensitivity or conversely).

The AUC criteria is then calculated on S1_{V}.A for each of the 23653 variables (PresenceAbsence R package), and the top 2000 variables (ranked by decreasing order of AUC−2 sd) were then selected for the further steps.

A distance matrix between these 2000 variables has then been calculated as 1−pearson correlation coefficient, using S1_{V}.A. A hierarchical clustering has then been performed on this distance matrix and the obtained dendrogram is cut in 50 clusters. In each cluster, the variable yielding the higher value of AUC−2 sd (obtained at the previous step) was kept.

These 50 genes were then used in a stepwise procedure to build multivariate models on S1_{V}. For a given combination of predictive variables, 3 algorithms (DLDA, DQDA, PAM) are trained on S1_{V}.A, yielding 3 predictors, which are then used to predict S1_{V}.B. The criterion is then calculated for each of the 3 predictors independently on S1_{V}.A and S1_{V}.B. Criterion values are then averaged over the 3 predictors and the current model was said superior to competitor models if it does as good as them on S1_{V}.A and better on S1_{V}.B.

A modified stepwise forward procedure was used: at run k>2 (i.e. building a model at k variables, based on a previously obtained model at (k−1) variables), a variable is added, then a variable is removed and a variable is added again. The variable to be added or removed is selected among those optimizing the criterion. When several variables are optimizing the criterion, the first encountered is selected. 15 models were built, ranging from 1 to 15 genes. The smallest model, i.e. with the less possible variables, optimizing the criterion, was then selected. To validate this model, it was used to predict samples from the validation set S2_{V}. As 3 algorithms are used in the model, a majority rule is used to get a unique class membership.

### Statistical Analysis

Continuous and discontinuous variable were compared using Mann Whitney and Chi square or fisher exact test respectively. Univariate and multivariate analysis were performed using the Cox model. Statistical analysis was performed using the R statistical software and rms package.

### Results

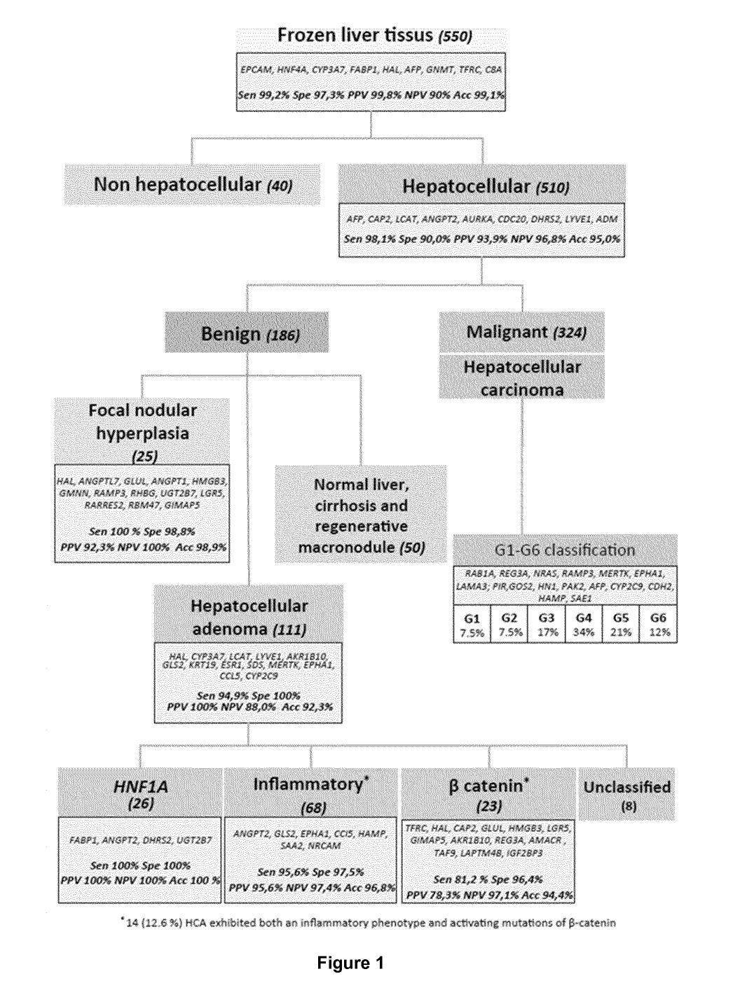

A molecular algorithm was constructed for diagnosis as a hierarchic tool used in a decisional tree (see FIG. 1).

The expression level of all the 103 selected genes was analyzed by quantitative RT-PCR. In the overall series of 550 included samples, each subgroup of samples were randomly separated (ratio 1/1) in a training and validation set in order to create and validate the molecular algorithm, respectively. Using a step-by-step analysis, 55 genes have been identified (described in Table 2) that could classify samples in each specific subgroups using a consensus between 3 nearest centroid methods (DLDA, DLQA and PAM, as detailed in Patients and Methods). Then, the robustness of the molecular classifiers was tested in the validation set of tumors (as described in FIG. 1 and in Table 3 below).

TABLE 3
accuracy of the molecular algorithm for the diagnosis of hepatocellular tumors
among 550 liver samples

| | Training | | | | | | Validation | | | | | | Training + validation | | | | | |
|---|---|---|---|---|---|---|---|---|---|---|---|---|---|---|---|---|---|---|
| | | Sen | Spe | PPV | NPV | Acc | | Sen | Spe | PPV | NPV | Acc | | Sen | Spe | PPV | NPV | Acc |
| | n | (%) | (%) | (%) | (%) | (%) | n | (%) | (%) | (%) | (%) | (%) | n | (%) | (%) | (%) | (%) | (%) |
| Non | 21/285 | 99.3 | 94.4 | 99.7 | 89.5 | 99.0 | 19/225 | 99.1 | 100 | 100 | 90.5 | 99.2 | 40/510 | 99.2 | 97.3 | 99.8 | 90.0 | 99.1 |
| hepato- | | | | | | | | | | | | | | | | | | |
| cellular/ | | | | | | | | | | | | | | | | | | |
| Hepato- | | | | | | | | | | | | | | | | | | |
| cellular | | | | | | | | | | | | | | | | | | |
| HCC/ | 191/96 | 97.9 | 96.8 | 98.4 | 95.8 | 97.6 | 133/90 | 98.3 | 84.8 | 87.2 | 97.8 | 91.5 | 324/186 | 98.1 | 90.0 | 93.8 | 96.8 | 94.9 |
| benign | | | | | | | | | | | | | | | | | | |
| hepato- | | | | | | | | | | | | | | | | | | |
| cellular | | | | | | | | | | | | | | | | | | |
| tissues | | | | | | | | | | | | | | | | | | |
| FNH/ | 13/83 | 100 | 100 | 100 | 100 | 100 | 12/78 | 100 | 97.5 | 83.4 | 100 | 97.7 | 25/161 | 100 | 98.8 | 92.3 | 100 | 98.9 |
| others | | | | | | | | | | | | | | | | | | |
| benign | | | | | | | | | | | | | | | | | | |
| tissues | | | | | | | | | | | | | | | | | | |
| HCA/ | 56/37 | 93.3 | 100 | 100 | 84.7 | 95.1 | 55/38 | 96.5 | 100 | 100 | 91.7 | 97.5 | 111/75 | 94.9 | 100 | 100 | 88 | 96.3 |
| others | | | | | | | | | | | | | | | | | | |
| benign | | | | | | | | | | | | | | | | | | |
| tissues | | | | | | | | | | | | | | | | | | |
| HNF1A | 13/43 | 100 | 100 | 100 | 100 | 100 | 13/42 | 100 | 100 | 100 | 100 | 100 | 26/85 | 100 | 100 | 100 | 100 | 100 |
| HCA/ | | | | | | | | | | | | | | | | | | |
| others | | | | | | | | | | | | | | | | | | |
| HCA | | | | | | | | | | | | | | | | | | |
| Inflam- | 34/22 | 100 | 92.3 | 93.8 | 100 | 96.4 | 34/21 | 97.2 | 94.7 | 97.2 | 94.7 | 96.4 | 68/43 | 98.5 | 93.3 | 95.6 | 97.7 | 96.4 |
| matory | | | | | | | | | | | | | | | | | | |
| HCA/ | | | | | | | | | | | | | | | | | | |
| others | | | | | | | | | | | | | | | | | | |
| HCA* | | | | | | | | | | | | | | | | | | |
| β | 12/44 | 84.6 | 95.3 | 95.3 | 92.9 | 95.1 | 11/44 | 77.8 | 93.3 | 70 | 95.5 | 90.7 | 23/88 | 81.8 | 94.3 | 78.3 | 95.4 | 91.8 |
| catenin | | | | | | | | | | | | | | | | | | |
| HCA/ | | | | | | | | | | | | | | | | | | |
| others | | | | | | | | | | | | | | | | | | |
| HCA* | | | | | | | | | | | | | | | | | | |

*14 (12.6%) HCA exhibited both an inflammatory phenotype and activating mutations of β-catenin
Benign hepatocellular tissus (n = 186) are composed of FNH (n = 25), HCA (n = 111), normal liver (n = 7), cirrhosis (n = 23, etiology: HCV n = 10, HBV n = 3, Alcohol n = 7, NASH n = 1, primary biliary cirrhosis n = 1, alpha-1 antitrypsin deficiency n = 1), non-dysplastic regenerative macronodule (n = 5) and dysplastic macronodule (n = 15).
Sen = sensitivity,
Spe = specificity,
PPV = positive predictive value,
NPV = negative predictive value,
Acc = accuracy,
HCC = hepatocellular carcinoma,
FNH = focal nodular hyperplasia,
HCA = hepatocellular adenoma

First, hepatocellular samples were efficiently identified from non-hepatocellular tumors by combining 9 genes (EPCAM, HNF4A, CYP3A7, FABP1, HAL, AFP, GNMT, TFRC, and C8A, see FIG. 1), then, benign hepatocellular samples were discriminated from HCC using a combination of 9 genes (AFP, CAP2, LCAT, ANGPT2, AURKA, CDC20, DHRS2, LYVE1, and ADM, see FIG. 1). HCC were also classified using the G1-G6 classification previously described in WO2007/063118A1, which permitted to confirm the reliability of this method in a large cohort of HCC, and the relationships previously described with the genetic and clinical features (see Table 4 below).

TABLE 4
Clinical and genetic features associated with G1-G6 classification
in HCC included in the diagnostic study (n = 324)

| | | Associated | Fisher exact test P |
|---|---|---|---|
| | | group | value in 324 HCC^{a} |
| Clinical | Female | G1 | 0.0086 |
| variables | HBV | G1-G2 | 0.0002 |
| | Age < 60 years old | G1-G2 | 0.0001 |
| | AFP > 20 ng/ml | G1 | <0.0001 |
| | Poor prognosis | G3 | <0.0001 |
| Genetic | TP53 mutations | G2-G3 | <0.0001 |
| alteration | CTNNB1 mutations | G5-G6 | <0.0001 |

^{a}Except for prognosis (n = 314)

Then, focusing on the benign subtypes of hepatocellular tumors, it was possible to identify HCA or FNH from the other benign hepatocellular tissues (including regenerative macronodule, dysplastic macronodule and non-tumor liver tissues) using 13 genes for FNH (HAL, ANGPTL7, GLUL, ANGPT1, HMGB3, GMNN, RAMP3, RHBG, UGT2B7, LGR5, RARRES2, RBM47, and GIMAP5, see FIG. 1) and 13 genes for HCA (HAL, CYP3A7, LCAT, LYVE1, AKR1B10, GLS2, KRT19, ESR1, SDS, MERTK, EPHA1, CCL5, and CYP2C9, see FIG. 1).

Finally, the different subtypes of HCA we classified: HNF1A mutated (4 genes: FABP1, ANGPT2, DHRS2, and UGT2B7, see FIG. 1), β catenin mutated (13 genes: TFRC, HAL, CAP2, GLUL, HMGB3, LGR5, GIMAP5, AKR1B10, REG3A, AMACR, TAF9, LAPTM4B, and IGF2BP3, see FIG. 1), and inflammatory adenomas (7 genes: ANGPT2, GLS2, EPHA1, CCI5, HAMP, SAA2, and NRCAM, see FIG. 1).

As shown in Table 3 above, for each type of tumors, more than 90% were obtained for sensitivity, specificity, negative predictive value, positive predictive value and accuracy in almost each branch of the diagnosis tree in both the training and validation set. These data underline the robustness of the 55 genes classification/diagnosis algorithm according to the invention.

### CONCLUSION

In this study, a molecular 55-genes algorithm has been identified and validated for the first time to classify both benign and malignant hepatocellular tumors in specific subgroups. In the diagnostic field of hepatocellular tumors, previous study have focused on diagnosis of early HCC, HCA or FNH but they have never captured the whole body of benign and malignant hepatocellular neoplasms (Bioulac Sage P hepatology 2007, Rebouissou S J hepatol 2008, Llovet J M gastroenterology 2006). In difficult cases, the algorithm according to the invention could help the pathological diagnosis by assessing the molecular subclass.

The 16 genes of the G1-G6 classification previously described in WO2007/063118A1 were also kept in the general algorithm, because different molecular subgroups constitute different potential therapeutic targets (G1 with IGFR1 inhibitor, G1-G2 with mTor inhibitor and G5-G6 with wnt inhibitor) and it could guide future clinical trial.

In conclusion, this study constitutes a new step in personalized medicine by providing a classification/diagnosis molecular algorithm to perform a global assessment of liver samples. This may help oncologists to take their therapeutic decisions for patients suspected to suffer from a liver tumor.

### BIBLIOGRAPHIC REFERENCES

Benhamouche S, Decaens T, Godard C, et al. Apc tumor suppressor gene is the “zonation-keeper” of mouse liver. Developmental cell 2006; 10:759-70.
Bioulac-Sage P, Rebouissou S, Thomas C, et al. Hepatocellular adenoma subtype classification using molecular markers and immunohistochemistry. Hepatology 2007; 46:740-8.
Bioulac-Sage P, Cubel G, Balabaud C, Zucman-Rossi J. Revisiting the pathology of resected benign hepatocellular nodules using new immunohistochemical markers. Seminars in liver disease 2011; 31:91-103.
Boyault S, Rickman D S, de Reynies A, et al. Transcriptome classification of HCC is related to gene alterations and to new therapeutic targets. Hepatology 2007; 45:42-52.
Cadoret A, Ovejero C, Terris B, et al. New targets of beta-catenin signaling in the liver are involved in the glutamine metabolism. Oncogene 2002; 21:8293-301.
Capurro M, Wanless I R, Sherman M, et al. Glypican-3: a novel serum and histochemical marker for hepatocellular carcinoma. Gastroenterology 2003; 125:89-97.
Chuma M, Sakamoto M, Yamazaki K, et al. Expression profiling in multistage hepatocarcinogenesis: identification of HSP70 as a molecular marker of early hepatocellular carcinoma. Hepatology 2003; 37:198-207.
El-Serag H B. Hepatocellular carcinoma. The New England journal of medicine 2011; 365:1118-27.
Forner A, Llovet J M, Bruix J. Hepatocellular carcinoma. Lancet 2012; 379:1245-55.
Forner A, Vilana R, Ayuso C, et al. Diagnosis of hepatic nodules 20 mm or smaller in cirrhosis: Prospective validation of the noninvasive diagnostic criteria for hepatocellular carcinoma. Hepatology 2008; 47:97-104.
Terminology of nodular hepatocellular lesions. International Working Party. Hepatology 1995; 22:983-93.
Pathologic diagnosis of early hepatocellular carcinoma: a report of the international consensus group for hepatocellular neoplasia. Hepatology 2009; 49:658-64.
Kondoh N, Wakatsuki T, Ryo A, et al. Identification and characterization of genes associated with human hepatocellular carcinogenesis. Cancer research 1999; 59:4990-6.
Lee J S, Heo J, Libbrecht L, et al. A novel prognostic subtype of human hepatocellular carcinoma derived from hepatic progenitor cells. Nature medicine 2006; 12:410-6.
Llovet J M, Chen Y, Wurmbach E, et al. A molecular signature to discriminate dysplastic nodules from early hepatocellular carcinoma in HCV cirrhosis. Gastroenterology 2006; 131:1758-67.
Odom D T, Zizlsperger N, Gordon D B, et al. Control of pancreas and liver gene expression by HNF transcription factors. Science 2004; 303:1378-81.
Ouelette and Bzevanis Bioinformatics: A Practical Guide for Analysis of Gene and Proteins (Wiley & Sons, Inc., 2^{nd} ed., 2001)
Paradis V, Bieche I, Dargere D, et al. A quantitative gene expression study suggests a role for angiopoietins in focal nodular hyperplasia. Gastroenterology 2003; 124:651-9.
Rashidi and Buehler, Bioinformatics Basics: Application in Biological Science and Medicine (CRC Press, London, 2000)
Rebouissou S, Imbeaud S, Balabaud C, et al. HNF1alpha inactivation promotes lipogenesis in human hepatocellular adenoma independently of SREBP-1 and carbohydrate-response element-binding protein (ChREBP) activation. The Journal of biological chemistry 2007; 282:14437-46.
Rebouissou S, Couchy G, Libbrecht L, et al. The beta-catenin pathway is activated in focal nodular hyperplasia but not in cirrhotic FNH-like nodules. Journal of hepatology 2008; 49:61-71.
Rebouissou S, Amessou M, Couchy G, et al. Frequent in-frame somatic deletions activate gp130 in inflammatory hepatocellular tumours. Nature 2009; 457:200-4.
Salzberg, Searles, Kasif, (Ed.), Computational Methods in Molecular Biology, (Elsevier, Amsterdam, 1998);
Setubal and Meidanis et al., Introduction to Computational Biology Methods (PWS Publishing Company, Boston, 1997);
Tsunedomi R, Iizuka N, Hamamoto Y, et al. Patterns of expression of cytochrome P450 genes in progression of hepatitis C virus-associated hepatocellular carcinoma. International journal of oncology 2005; 27:661-7.
van Aalten S M, Verheij J, Terkivatan T, Dwarkasing R S, de Man R A, Ijzermans J N. Validation of a liver adenoma classification system in a tertiary referral centre: implications for clinical practice. Journal of hepatology 2011; 55:120-5. WO2007/063118A1
Yamamoto Y, Sakamoto M, Fujii G, et al. Overexpression of orphan G-protein-coupled receptor, Gpr49, in human hepatocellular carcinomas with beta-catenin mutations. Hepatology 2003; 37:528-33.
Yamashita T, Forgues M, Wang W, et al. EpCAM and alpha-fetoprotein expression defines novel prognostic subtypes of hepatocellular carcinoma. Cancer research 2008; 68:1451-61.
Zucman-Rossi J, Jeannot E, Nhieu J T, et al. Genotype-phenotype correlation in hepatocellular adenoma: new classification and relationship with HCC. Hepatology 2006; 43:515-24.

1. A method for classifying in vitro a liver sample as a non-hepatocellular sample, a hepatocellular carcinoma (HCC) sample, a focal nodule dysplasia (FNH) sample, a hepatocellular adenoma (HCA) sample or another benign liver sample, comprising:
a) Determining in vitro from said liver sample an expression profile comprising the 38 following genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9;
b) Determining if said liver sample is a hepatocellular or a non-hepatocellular sample, based on the expression levels measured for an expression profile comprising the 9 following genes: EPCAM, HNF4A, CYP3A7, FABP1, HAL, AFP, GNMT, TFRC, and C8A, using at least one algorithm calibrated with at least one reference liver sample;
c) If said liver sample is a hepatocellular sample, then determining if said hepatocellular sample is a HCC sample or a benign hepatocellular sample, based on the expression levels measured for an expression profile comprising the 9 following genes: AFP, CAP2, LCAT, ANGPT2, AURKA, CDC20, DHRS2, LYVE1, and ADM, using at least one algorithm calibrated with at least one reference liver sample;
d) If said liver sample is a benign hepatocellular sample, then determining if said benign hepatocellular sample is a FNH sample, based on the expression levels measured for an expression profile comprising the 13 following genes: HAL, ANGPTL7, GLUL, ANGPT1, HMGB3, GMNN, RAMP3, RHBG, UGT2B7, LGR5, RARRES2, RBM47, and GIMAP5, using at least one algorithm calibrated with at least one reference liver sample;
e) If said liver sample is a benign hepatocellular sample, then determining if said benign hepatocellular sample is a HCA sample, based on the expression levels measured for an expression profile comprising the 13 following genes: HAL, CYP3A7, LCAT, LYVE1, AKR1B10, GLS2, KRT19, ESR1, SDS, MERTK, EPHA1, CCL5, and CYP2C9, using at least one algorithm calibrated with at least one reference liver sample;
f) If said benign hepatocellular sample is neither a FNH sample nor a HCA sample, then it is classified as another benign liver sample.

2. The method of claim 1, further comprising, if the liver sample is diagnosed as a HCA sample, classifying said HCA sample into one of the following HCA subgroups: HNF1A mutated HCA, inflammatory HCA, β catenin mutated HCA or other HCA, by:
a) Further determining in vitro from said HCA sample an expression profile comprising the 8 additional following genes: HAMP, SAA2, NRCAM, REG3A, AMACR, TAF9, LAPTM4B, and IGF2BP3;
b) Determining if said HCA sample is or not a HNF1A mutated HCA sample, based on the expression levels measured for an expression profile comprising the 4 following genes: FABP1, ANGPT2, DHRS2, and UGT2B7, using at least one algorithm calibrated with at least one reference liver sample;
c) Determining if said HCA sample is or not an inflammatory HCA sample, based on the expression levels measured for an expression profile comprising the 7 following genes: ANGPT2, GLS2, EPHA1, CCI5, HAMP, SAA2, and NRCAM, using at least one algorithm calibrated with at least one reference liver sample;
d) Determining if said HCA sample is or not a β catenin mutated HCA sample, based on the expression levels measured for an expression profile comprising the 13 following genes: TFRC, HAL, CAP2, GLUL, HMGB3, LGR5, GIMAP5, AKR1B10, REG3A, AMACR, TAF9, LAPTM4B, and IGF2BP3, using at least one algorithm calibrated with at least one reference liver sample;
e) If said HCA sample is neither a HNF1A mutated HCA sample, an inflammatory HCA sample, nor a β catenin mutated HCA sample, then it is classified as another HCA sample.

3. The method according to claim 1, further comprising, if the liver sample is diagnosed as a HCC sample, classifying said HCC sample into one of subgroups G1 to G6 defined by the following clinical and genetic main features:

| | G1 | G2 | G3 | G4 | G5 | G6 |
|---|---|---|---|---|---|---|
| Chromosome instability | + | + | + | − | − | − |
| Early relapse and death | + | + | + | − | − | − |
| TP53 mutation | − | + | + | − | − | − |
| HBV infection | + | + | − | − | − | − |
| Low copy number | + | − | − | − | − | − |
| High copy number | − | + | − | − | − | − |
| CTNNB1 mutation | − | − | − | − | + | + |
| Satellite nodules | − | − | − | − | − | + |

wherein classification is made by:
a) Further determining in vitro from said HCC sample an expression profile comprising or consisting of the 11 additional following genes: RAB1A, REG3A, NRAS, PIR, LAMA3, G0S2, HN1, PAK2, CDH2, HAMP, and SAE1; and
b) calculating 6 subgroup distances based on the expression levels measured for an expression profile comprising or consisting of the 16 following genes: RAB1A, REG3A, NRAS, RAMP3, MERTK, PIR, EPHA1, LAMA3, G0S2, HN1, PAK2, AFP, CYP2C9, CDH2, HAMP, and SAE1; and
c) classifying said HCC tumor in the subgroup for which the subgroup distance is the lowest.

4. The method according to claim 1, wherein reference samples used for calibrating algorithms used for interpreting each expression profile are the following:
a) For determining if a liver sample is or not a hepatocellular sample: at least one hepatocellular sample and at least one non-hepatocellular sample;
b) For determining if a hepatocellular sample is or not a HCC sample: at least one benign sample and at least one HCC sample;
c) For determining if a benign hepatocellular sample is or not a FNH sample: at least one FNH sample and at least one non-FNH benign hepatocellular sample;
d) For determining if a benign hepatocellular sample is or not a HCA sample: at least one HCA sample and at least one non-HCA benign hepatocellular sample;
e) For determining if a HCA sample is or not a HNF1A mutated HCA sample: at least one HNF1A mutated HCA sample and at least one non-HNF1A mutated HCA sample;
f) For determining if a HCA sample is or not an inflammatory HCA sample: at least one inflammatory HCA sample and at least one non-inflammatory HCA sample;
g) For determining if a HCA sample is or not a β catenin mutated HCA sample: at least one β catenin mutated HCA sample and at least one non-β catenin mutated HCA sample; and
h) For classifying a HCC sample into one of subgroups G1 to G6: at least one sample of each G1 to G6 subgroups.

5. The method according to claim 1, wherein said liver sample is a liver biopsy or a partial or whole liver tumor surgical resection.

6. The method according to claim 1, wherein said expression profile(s) is(are) determined at the nucleic level.

7. The method according to claim 6, wherein said expression profile(s) is(are) determined using quantitative PCR.

8. The method according to claim 1, wherein the algorithm(s) used for interpreting any expression profile are selected from:
a) Prediction Analysis of Microarrays (PAM):
PAM(sample X)=Arg max(θ_{yes}(sample X);θ_{No}(sample X))
wherein
$${θ}_{Yes} ( sample X ) = ( {\sum }_{i = 1}^{N} \frac{( {x}_{i} - {π}_{i} )}{{γ}_{i}} \times {π}_{Yes , i} ) - {K}_{Yes}$$
$${θ}_{No} ( sample X ) = ( {\sum }_{i = 1}^{N} \frac{( {x}_{i} - {π}_{i} )}{{γ}_{i}} \times {π}_{No , i} ) - {K}_{No}$$
wherein:
xᵢ, 1≦i≦N, represent the in vitro measured values of N variables derived from the expression levels of genes of the expression profile, and
πᵢ, γᵢ, π_{Yes,i}, π_{No,i}, 1≦i≦N, K_{Yes} and K_{No} are fixed parameters calibrated with at least one reference sample;
b) Diagonal Linear Discriminant Analysis (DLDA):
DLDA(sample X)=Arg min(Δ_{Yes}(sample X);Δ_{No}(sample X))
wherein
$${Δ}_{Yes} ( sample X ) = {\sum }_{i = 1}^{N} \frac{{( {x}_{i} - {μ}_{yes , i} )}^{2}}{{υ}_{i}}$$
$${Δ}_{No} ( sample X ) = {\sum }_{i = 1}^{N} \frac{{( {x}_{i} - {μ}_{No , i} )}^{2}}{{υ}_{i}}$$
wherein:
xᵢ, 1≦i≦N, represent the in vitro measured values of N variables derived from the expression levels of genes of the expression profile, and
uᵢ, μ_{Yes,i}, and μ_{No,i}, 1≦i≦N, are fixed parameters calibrated with at least one reference sample;
c) Diagonal quadratic discriminant analysis (DQDA):
DQDA(sample X)=Arg min(∇_{Yes}(sample X);∇_{No}(sample X))
wherein
$${▽}_{Yes} ( sample X ) = ( {\sum }_{i = 1}^{N} \frac{{( {x}_{i} - {μ}_{Yes , i} )}^{2}}{{v}_{Yes , i}} ) + {C}_{Yes}$$
$${▽}_{No} ( sample X ) = ( {\sum }_{i = 1}^{N} \frac{{( {x}_{i} - {μ}_{No , i} )}^{2}}{{v}_{No , i}} ) + {C}_{No}$$
wherein:
xᵢ, 1≦i≦N, represent the in vitro measured values of N variables derived from the expression levels of genes of the expression profile, and
u_{Yes,i}, u_{No}, μ_{Yes,i}, μ_{No,i}, 1≦i≦N, are fixed parameters calibrated with at least one reference sample, and
$${C}_{Yes} = ( {\sum }_{i = 1}^{N} log ( {v}_{Yes , i} ) )$$
$${C}_{No} = ( {\sum }_{i = 1}^{N} log ( {v}_{No , i} ) ) ;$$
d) or any combination thereof.

9. The method of claim 8, wherein the algorithm used for interpreting each expression profile is:
Diagnosis(sample X)=majority rule(PAM(sample X),DLDA(sample X),DQDA(sample X)).

10. The method according to claim 9, wherein said expression profile(s) is(are) determined using quantitative PCR and the variables and parameters of PAM, DLDA and DQDA algorithms are the following:
a) For determining if a liver sample is or not a hepatocellular sample:
6 variables x₁ to x₆ are used as follows:

| | |
|---|---|
| x₁ | (−ΔΔCt TFRC expression level) − (−ΔΔCt C8A expression level) |
| x₂ | (−ΔΔCt AFP expression level) + (−ΔΔCt GNMT expression level) |
| x₃ | (−ΔΔCt HAL expression level) − (−ΔΔCt EPCAM expression level) |
| x₄ | (−ΔΔCt CYP3A7 expression level) − (−ΔΔCt EPCAM expression level) |
| x₅ | (−ΔΔCt FABP1 expression level) − (−ΔΔCt EPCAM expression level) |
| x₆ | (−ΔΔCt EPCAM expression level) − (−ΔΔCt HNF4A expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | 1.342931 | −0.09325912 | 2.006058 | 7.153821 | 8.151418 | 0.0932632 |
| x₂ | −1.551583 | 0.10774885 | −4.1733248 | 9.685958 | | |
| x₃ | −1.23594 | 0.08582914 | −0.9310016 | 10.17258 | | |
| x₄ | −1.524252 | 0.10585085 | 2.8897574 | 10.391148 | | |
| x₅ | −1.261254 | 0.08758709 | −1.0531553 | 10.049158 | | |
| x₆ | 1.087001 | −0.07548619 | −1.4702021 | 9.901341 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | 11.613149 | 1.3388989 | 11.690171 | 4.251989 | 4.692407 |
| x₂ | −19.201897 | −3.12967394 | 12.73627 | 22.662048 | 22.074337 |
| x₃ | −13.503695 | −0.05789783 | 17.965523 | 27.445047 | 26.883759 |
| x₄ | −12.948974 | 3.98966931 | 6.765985 | 30.609874 | 29.198065 |
| x₅ | −13.727697 | −0.17297876 | 17.267584 | 26.144739 | 25.619118 |
| x₆ | 9.292567 | −2.21761661 | 1.913791 | 25.543753 | 24.14461 |

b) For determining if a hepatocellular sample is or not a HCC sample:
6 variables x₁ to x₆ are used as follows:

| | |
|---|---|
| x₁ | (−ΔΔCt CAP2 expression level) − (−ΔΔCt LCAT expression level) |
| x₂ | (−ΔΔCt ANGPT2 expression level) + (−ΔΔCt AURKA expression level) |
| x₃ | (−ΔΔCt CDC20 expression level) + (−ΔΔCt DHRS2 expression level) |
| x₄ | (−ΔΔCt ANGPT2 expression level) − (−ΔΔCt LYVE1 expression level) |
| x₅ | (−ΔΔCt ADM expression level) − (−ΔΔCt CDC20 expression level) |
| x₆ | Max (−ΔΔCt AFP expression level; −ΔΔCt CAP2 expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.16268042 | 0.08134021 | 5.787048 | 4.542418 | 1.272916 | 0.449041 |
| x₂ | −0.22453753 | 0.11226876 | 3.035909 | 3.975872 | | |
| x₃ | −0.42378458 | 0.21189229 | 3.937962 | 6.248688 | | |
| x₄ | −0.2592874 | 0.1296437 | 4.151425 | 3.70769 | | |
| x₅ | 0.15685585 | −0.07842792 | −4.403932 | 3.840179 | | |
| x₆ | −0.01726311 | 0.00863156 | 3.696066 | 4.123495 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | 2.678847 | 7.341149 | 2.2201 | 8.37556 | 6.33819 |
| x₂ | 0.06943705 | 4.519144 | 3.255149 | 4.0793 | 3.806517 |
| x₃ | −1.96933307 | 6.891609 | 25.818236 | 13.894186 | 17.840878 |
| x₄ | 1.25620635 | 5.599034 | 1.863177 | 3.311281 | 2.831979 |
| x₅ | −1.79861246 | −5.706591 | 2.246134 | 3.814584 | 3.295449 |
| x₆ | 1.47414444 | 4.807026 | 1.020023 | 6.078697 | 4.404347 |

c) For determining if a benign hepatocellular sample is or not a FNH sample:
12 variables x₁ to x₁₂ are used as follows:

| | |
|---|---|
| x₁ | Min (−ΔΔCt ANGPTL7 expression level; −ΔΔCt GLUL expression |
| | level) |
| x₂ | (−ΔΔCt ANGPT1 expression level) − (−ΔΔCt HMGB3 expression level) |
| x₃ | (−ΔΔCt GMNN expression level) + (−ΔΔCt RAMP3 expression level) |
| x₄ | Min (−ΔΔCt RHBG expression level; −ΔΔCt UGT2B7 expression level) |
| x₅ | Max (−ΔΔCt HAL expression level; −ΔΔCt RAMP3 expression level) |
| x₆ | Min (−ΔΔCt LGR5 expression level; −ΔΔCt UGT2B7 expression level) |
| x₇ | (−ΔΔCt RAMP3 expression level) + (−ΔΔCt UGT2B7 expression level) |
| x₈ | (−ΔΔCt RAMP3 expression level) + (−ΔΔCt RARRES2 expression |
| | level) |
| x₉ | Max (−ΔΔCt ANGPT1 expression level; −ΔΔCt RAMP3 expression |
| | level) |
| x₁₀ | Min (−ΔΔCt ANGPT1 expression level; −ΔΔCt LGR5 expression level) |
| x₁₁ | (−ΔΔCt RAMP3 expression level) − (−ΔΔCt RBM47 expression level) |
| x₁₂ | Min (−ΔΔCt GIMAP5 expression level; −ΔΔCt UGT2B7 expression |
| | level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.18469273 | 1.0817717 | −1.72829395 | 3.243668 | 0.2800792 | 6.1260851 |
| x₂ | −0.15724871 | 0.9210281 | 0.61243528 | 2.336453 | | |
| x₃ | −0.13637923 | 0.7987926 | 1.58326744 | 2.289755 | | |
| x₄ | −0.15358836 | 0.899589 | −3.46104209 | 3.909901 | | |
| x₅ | −0.11234999 | 0.65805 | 1.19490255 | 2.017152 | | |
| x₆ | −0.11945816 | 0.6996835 | −2.27683325 | 3.334501 | | |
| x₇ | −0.15338781 | 0.8984143 | −0.04692744 | 2.922347 | | |
| x₈ | −0.14256206 | 0.8350063 | 0.60258802 | 2.277919 | | |
| x₉ | −0.11634108 | 0.6814263 | 1.54744785 | 1.913217 | | |
| x₁₀ | −0.17351058 | 1.0162762 | −1.4122167 | 3.581967 | | |
| x₁₁ | −0.15477031 | 0.9065118 | 1.45598643 | 2.048925 | | |
| x₁₂ | −0.07438928 | 0.4357086 | −1.04952428 | 2.524675 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | −2.3273759 | 1.7806145 | 4.6402628 | 0.60826433 | 4.11435 |
| x₂ | 0.245031 | 2.76437457 | 1.4145492 | 0.20686229 | 1.2570248 |
| x₃ | 1.2709924 | 3.41230679 | 1.2978397 | 0.19883833 | 1.1544917 |
| x₄ | −4.0615574 | 0.05626186 | 8.3471726 | 0.0196296 | 7.2609714 |
| x₅ | 0.9682756 | 2.52228907 | 0.6935121 | 0.30621156 | 0.6429946 |
| x₆ | −2.6751666 | 0.05626186 | 5.1618051 | 0.0196296 | 4.4910865 |
| x₇ | −0.4951798 | 2.57855093 | 3.3012094 | 0.33314121 | 2.9140701 |
| x₈ | 0.2778432 | 2.50466495 | 1.2384457 | 0.40087507 | 1.1291973 |
| x₉ | 1.3248621 | 2.85116431 | 0.5424233 | 0.11837803 | 0.487113 |
| x₁₀ | −2.0337258 | 2.22805082 | 6.3954525 | 0.30614496 | 5.601195 |
| x₁₁ | 1.1388737 | 3.31336105 | 0.7211325 | 0.52047864 | 0.6949603 |
| x₁₂ | −1.2373331 | 0.05049854 | 1.9692555 | 0.01620956 | 1.7145104 |

d) For determining if a benign hepatocellular sample is or not a HCA sample:
10 variables x₁ to x₁₀ are used as follows:

| | |
|---|---|
| x₁ | (−ΔΔCt AKR1B10 expression level) + (−ΔΔCt GLS2 expression level) |
| x₂ | (−ΔΔCt LCAT expression level) − (−ΔΔCt KRT19 expression level) |
| x₃ | (−ΔΔCt ESR1 expression level) + (−ΔΔCt SDS expression level) |
| x₄ | Max (−ΔΔCt MERTK expression level; −ΔΔCt LYVE1 expression |
| | level) |
| x₅ | Max (−ΔΔCt EPHA1 expression level; −ΔΔCt KRT19 expression level) |
| x₆ | (−ΔΔCt CCL5 expression level) + (−ΔΔCt GLS2 expression level) |
| x₇ | (−ΔΔCt HAL expression level) − (−ΔΔCt MERTK expression level) |
| x₈ | (−ΔΔCt CYP2C9 expression level) − (−ΔΔCt MERTK expression level) |
| x₉ | (−ΔΔCt CCL5 expression level) + (−ΔΔCt KRT19 expression level) |
| x₁₀ | Min (−ΔΔCt CYP3A7 expression level; −ΔΔCt EPHA1 expression |
| | level) |

PAM parameters are the following:

| xᵢ | π_{No,i} | π_{Yes,i} | πᵢ | γᵢ | K_{No} | K_{Yes} |
|---|---|---|---|---|---|---|
| x₁ | 1.1300586 | −0.52467006 | −0.96573089 | 5.405409 | 3.0655113 | 0.7945744 |
| x₂ | −0.6257754 | 0.29053858 | 0.10777331 | 4.174906 | | |
| x₃ | −0.583684 | 0.27099612 | 1.53413349 | 3.92968 | | |
| x₄ | −0.2101061 | 0.09754928 | 0.01545178 | 2.53848 | | |
| x₅ | 0.4031816 | −0.18719147 | 0.76400666 | 2.906802 | | |
| x₆ | 0.6342941 | −0.29449369 | −1.82990856 | 4.756332 | | |
| x₇ | 0.5211003 | −0.24193944 | −0.57174662 | 4.026102 | | |
| x₈ | 0.3773559 | −0.17520095 | −0.97286634 | 3.529012 | | |
| x₉ | 0.8070427 | −0.3746984 | −0.75070901 | 3.946451 | | |
| x₁₀ | 0.3875215 | −0.17992069 | 0.02720304 | 2.927056 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | 5.142698 | −3.8017871 | 1.9223207 | 16.202619 | 11.8086811 |
| x₂ | −2.5047803 | 1.3207446 | 4.8696186 | 4.8642148 | 4.8658775 |
| x₃ | −0.75955 | 2.5990617 | 1.5948539 | 4.8438216 | 3.8441392 |
| x₄ | −0.5178985 | 0.2630787 | 0.1157701 | 0.4169368 | 0.3242701 |
| x₅ | 1.9359758 | 0.2198781 | 0.9741474 | 0.8373057 | 0.8794108 |
| x₆ | 1.1870048 | −3.2306184 | 0.5402267 | 10.9818415 | 7.769037 |
| x₇ | 1.5262567 | −1.5458196 | 1.0506355 | 5.6452689 | 4.2315355 |
| x₈ | 0.358827 | −1.5911525 | 0.2637763 | 3.3978705 | 2.4335338 |
| x₉ | 2.4342454 | −2.2294378 | 3.9252834 | 3.9034702 | 3.910182 |
| x₁₀ | 1.1615001 | −0.4994349 | 0.507857 | 1.1000088 | 0.9178082 |

e) For determining if a HCA sample is or not a HNF1A mutated HCA sample:
2 variables x₁ to x₆ are used as follows:

| | |
|---|---|
| x₁ | (−ΔΔCt DHRS2 expression level) − (−ΔΔCt UGT2B7 expression level) |
| x₂ | (−ΔΔCt ANGPT2 expression level) + (−ΔΔCt FABP1 expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.2597076 | 1.817954 | −1.130125 | 6.501417 | 0.1803095 | 4.3715711 |
| x₂ | −0.1615805 | 1.131063 | 1.136677 | 3.83618 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | −2.8185929 | 10.68915 | 15.46252 | 14.3631833 | 15.343027 |
| x₂ | 0.5168253 | 5.47564 | 1.668767 | 0.7321017 | 1.566956 |

f) For determining if a HCA sample is or not an inflammatory HCA sample:
4 variables x₁ to x₆ are used as follows:

| | |
|---|---|
| x₁ | (−ΔΔCt HAMP expression level) + (−ΔΔCt SAA2 expression level) |
| x₂ | (−ΔΔCt CCL5 expression level) − (−ΔΔCt NRCAM expression level) |
| x₃ | Max (−ΔΔCt EPHA1 expression level; −ΔΔCt KRT19 expression level) |
| x₄ | (−ΔΔCt ANGPT2 expression level) + (−ΔΔCt SAA2 expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | −0.4760712 | 0.9521423 | 4.6430007 | 6.107883 | 0.7344381 | 2.4145044 |
| x₂ | 0.434627 | −0.869254 | −0.0574931 | 5.002872 | | |
| x₃ | 0.1882468 | −0.3764937 | 1.1521703 | 3.158128 | | |
| x₄ | −0.4549338 | 0.9098677 | 4.5882009 | 4.501345 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | 1.735214 | 10.4585747 | 16.9585649 | 7.6603747 | 13.9265464 |
| x₂ | 2.11689 | −4.4062595 | 7.0569419 | 6.5761749 | 6.90017 |
| x₃ | 1.746678 | −0.0368447 | 0.7298408 | 0.3673544 | 0.6116387 |
| x₄ | 2.540387 | 8.6838292 | 4.4787841 | 4.5955546 | 4.5168614 |

g) For determining if a HCA sample is or not a β catenin mutated HCA sample:
9 variables x₁ to x₆ are used as follows:

| | |
|---|---|
| x₁ | (−ΔΔCt AKR1B10 expression level) − (−ΔΔCt REG3A expression |
| | level) |
| x₂ | (−ΔΔCt AMACR expression level) + (−ΔΔCt HAL expression level) |
| x₃ | (−ΔΔCt CAP2 expression level) − (−ΔΔCt GLUL expression level) |
| x₄ | (−ΔΔCt HAL expression level) + (−ΔΔCt TAF9 expression level) |
| x₅ | (−ΔΔCt CAP2 expression level) − (−ΔΔCt LGR5 expression level) |
| x₆ | Min (−ΔΔCt AKR1B10 expression level; −ΔΔCt HAL expression level) |
| x₇ | (−ΔΔCt LAPTM4B expression level) + (−ΔΔCt TFRC expression level) |
| x₈ | (−ΔΔCt GIMAP5 expression level) − (−ΔΔCt HAL expression level) |
| x₉ | (−ΔΔCt HMGB3 expression level) − (−ΔΔCt IGF2BP3 expression level) |

PAM parameters are the following:

| xᵢ | π_{No,i} | π_{Yes,i} | πᵢ | γᵢ | K_{No} | K_{Yes} |
|---|---|---|---|---|---|---|
| x₁ | 0.34708654 | −1.9668237 | 1.94438201 | 7.392962 | 0.3607787 | 8.2634614 |
| x₂ | 0.21863143 | −1.2389115 | −1.04516656 | 3.127947 | | |
| x₃ | 0.18579207 | −1.0528217 | 1.22379671 | 2.663529 | | |
| x₄ | 0.24406366 | −1.3830274 | 0.05214403 | 3.244264 | | |
| x₅ | 0.15694722 | −0.8893676 | 2.7521494 | 3.869139 | | |
| x₆ | 0.21470021 | −1.2166345 | −1.47714108 | 4.260375 | | |
| x₇ | 0.11140632 | −0.6313025 | 0.81968112 | 3.203963 | | |
| x₈ | −0.22080529 | 1.25123 | 0.49103172 | 3.193991 | | |
| x₉ | 0.04764503 | −0.2699885 | 0.56180483 | 3.025541 | | |

DLDA and DQDA parameters are the same, as follows:

| xᵢ | μ_{No, i} | μ_{Yes, i} | ∪_{No, i} | ∪_{Yes, i} | ∪ᵢ |
|---|---|---|---|---|---|
| x₁ | 4.5103796 | 12.5962709 | 37.671414 | 6.2381109 | 33.535453 |
| x₂ | −0.361299 | −4.920416 | 1.426277 | 8.2837077 | 2.328571 |
| x₃ | 1.7186592 | −1.5804241 | 1.203395 | 0.6218992 | 1.126882 |
| x₄ | 0.8439509 | −4.4347616 | 1.358794 | 11.5298442 | 2.69709 |
| x₅ | 3.3594 | −0.6889375 | 5.646265 | 1.7986761 | 5.140003 |
| x₆ | −0.5624378 | −6.6604599 | 6.819184 | 8.7029888 | 7.067053 |
| x₇ | 1.1766229 | −1.2029889 | 2.912529 | 0.2815287 | 2.566345 |
| x₈ | −0.2142184 | 4.4874493 | 1.580383 | 8.8316336 | 2.534495 |
| x₉ | 0.7059568 | −0.2550566 | 2.287403 | 0.3047094 | 2.026522 |

11. The method according to claim 3, wherein the HCC sample is classified into one of subgroups G1 to G6 using the following formula for calculating the distance of said HCC sample to each subgroup Gₖ, 1≦k≦6:
$${\sum }_{t = 1 … 16} \frac{\begin{matrix}Distance ( HCC sample , subgroup {G}_{k} ) = \\ {( Δ Ct ( HCC sample , subgroup {G}_{k} , {gene}_{t} ) - μ ( subgroup {G}_{k} , {gene}_{t} ) )}^{2}\end{matrix}}{σ ( {gene}_{t} )}$$
wherein for each geneₜ and subgroup Gₖ, the μ(subgroup Gₖ, geneₜ) and σ(geneₜ) values are the following:

| μ | G1 | G2 | G3 | G4 | G5 | G6 | σ |
|---|---|---|---|---|---|---|---|
| gene 1 (RAB1A) | −16.39 | −16.04 | −16.29 | −17.15 | −17.33 | −16.95 | 0.23 |
| gene 2 (PAP) | −28.75 | −27.02 | −23.48 | −27.87 | −19.23 | −11.33 | 16.63 |
| gene 3 (NRAS) | −16.92 | −17.41 | −16.25 | −17.31 | −16.96 | −17.26 | 0.27 |
| gene 4 | −23.54 | −23.12 | −25.34 | −22.36 | −23.09 | −23.06 | 1.23 |
| (RAMP3) | | | | | | | |
| gene 5 | −18.72 | −18.43 | −21.24 | −18.29 | −17.03 | −16.16 | 7.23 |
| (MERTK) | | | | | | | |
| gene 6 (PIR) | −18.44 | −19.81 | −16.73 | −18.28 | −17.09 | −17.25 | 0.48 |
| gene 7 (EPHA1) | −16.68 | −16.51 | −19.89 | −17.04 | −18.70 | −21.98 | 1.57 |
| gene 8 (LAMA3) | −20.58 | −20.44 | −20.19 | −21.99 | −18.77 | −16.85 | 2.55 |
| gene 9 (G0S2) | −14.82 | −17.45 | −18.18 | −14.78 | −17.99 | −16.06 | 3.88 |
| gene 10 (HN1) | −16.92 | −17.16 | −15.91 | −17.88 | −17.72 | −17.93 | 0.54 |
| gene 11 (PAK2) | −17.86 | −16.56 | −16.99 | −18.14 | −17.92 | −17.97 | 0.58 |
| gene 12 (AFP) | −16.68 | −12.36 | −26.80 | −27.28 | −25.97 | −23.47 | 14.80 |
| gene 13 | −18.27 | −16.99 | −16.26 | −16.23 | −13.27 | −14.44 | 5.47 |
| (CYP2C9) | | | | | | | |
| gene 14 (CDH2) | −15.20 | −14.76 | −18.91 | −15.60 | −15.48 | −17.32 | 10.59 |
| gene 15 | −19.53 | −20.19 | −21.32 | −18.51 | −25.06 | −26.10 | 13.08 |
| (HAMP) | | | | | | | |
| gene 16 (SAE1) | −17.37 | −17.10 | −16.79 | −18.22 | −17.72 | −18.16 | 0.31 |

12. A kit comprising reagents for the determination of an expression profile comprising at most 65 distinct genes, wherein said expression profile is selected from:
An expression profile comprising the following 38 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9;
An expression profile comprising the following 46 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, and IGF2BP3;
An expression profile comprising the following 49 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, RAB1A, REG3A, NRAS, PIR, LAMA3, G0S2, HN1, PAK2, CDH2, HAMP, and SAE1; or
An expression profile comprising the following 55 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, IGF2BP3, RAB1A, NRAS, PIR, LAMA3, G0S2, HN1, PAK2, CDH2.

13. The kit according to claim 12, comprising:
a) specific amplification primers pairs and/or probes, or
b) a nucleic acid microarray.

14. (canceled)

15. A system 1 for classifying a liver sample comprising:
a) a determination module 2 configured to receive a liver sample and to determine expression level information concerning:
An expression profile comprising the following 38 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, and CYP2C9;
An expression profile comprising the following 46 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, and IGF2BP3;
An expression profile comprising the following 49 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, RAB1A, REG3A, NRAS, PIR, LAMA3, G0S2, HN1, PAK2, CDH2, HAMP, and SAE1; or
An expression profile comprising the following 55 genes: EPCAM, HNF4A, CYP3A7, FABP1, HAL, AFP, GNMT, TFRC, C8A, CAP2, LCAT, ANGPT2, AURKA, CDC20, DHRS2, LYVE1, ADM, ANGPTL7, GLUL, ANGPT1, HMGB3, GMNN, RAMP3, RHBG, UGT2B7, LGR5, RARRES2, RBM47, GIMAP5, AKR1B10, GLS2, KRT19, ESR1, SDS, MERTK, EPHA1, CCL5, CYP2C9, HAMP, SAA2, NRCAM, REG3A, AMACR, TAF9, LAPTM4B, IGF2BP3, RAB1A, NRAS, PIR, LAMA3, G0S2, HN1, PAK2, CDH2, and SAE1;
b) a storage device 3 configured to store the expression level information from the determination module;
c) a comparison module 4, adapted to compare the expression level information stored on the storage device with reference data, and to provide a comparison result, wherein the comparison result is indicative of the type of liver sample; and
d) a display module 5 for displaying a content 6 based in part on the classification result for the user, wherein the content is a signal indicative of the type of liver sample.

16. A computer readable medium 7 having computer readable instructions recorded thereon to define software modules for implementing on a computer steps of a prognosis method according to claim 1 relating to interpretation of expression profiles data.

17. A method for treating a liver disease in a subject in need thereof, comprising:
a) Classifying a liver sample of said subject as a non-hepatocellular sample, a hepatocellular carcinoma (HCC) sample, a focal nodule dysplasia (FNH) sample, a hepatocellular adenoma (HCA) sample or another benign liver sample with the classification method according to claim 1;
b) If said sample is a non-hepatocellular sample, then identifying the precise histological subtype of sample and administering to said subject a treatment according to the histological subtype identified;
c) If said sample is a HCC sample, then performing surgical resection with or without adjuvant treatment;
d) If said sample is a FNH sample, then no therapeutic action is performed;
e) If said sample is a HCA sample, then only following up the subject or performing surgical resection, depending on the HCA subgroup;
f) If said sample is another benign hepatocellular sample, then no therapeutic action is performed.

18. The method according to claim 17, further comprising, if said liver sample is an HCC sample:
i. classifying said HCC sample into one of subgroups G1 to G6 according to the method of claim 3; and
ii. if said HCC sample is classified in G1 subgroup, then administering an efficient amount of an IGFR1 inhibitor or of an Akt/mTor inhibitor to said patient;
iii. if said HCC sample is classified in G1-G2 subgroup, administering an efficient amount of an hen Akt/mTor inhibitor to said patient;
iv. if said HCC sample is classified in G3 subgroup, then administering an efficient amount of a proteasome inhibitor to said patient;
v. if said HCC sample is classified in G5-G6 subgroup, then administering an efficient amount of a wnt inhibitor to said patient.

19. The method according to claim 17, further comprising, if said liver sample is an HCC sample:
i. Prognosing global survival and/or survival without relapse; and
ii. if said HCC sample is given a good prognosis, then no adjuvant treatment is performed;
iii. if said HCC sample is given a bad prognosis, then administering to said subject an adjuvant treatment.

20. The method according to claim 19, wherein said adjuvant treatment is selected from cytotoxic chemotherapy and/or targeted therapy.

21. The method according to claim 17, further comprising, if said liver sample is an HCA sample:
i. classifying said HCA sample into one of subgroups HNF1A mutated HCA, inflammatory HCA, β catenin mutated HCA or other HCA according to the method of claim 2; and
ii. if said HCA sample is classified as a HNF1A mutated HCA sample, then only following up said subject if HCA<5 cm, or performing surgical resection if HCA>5 cm;
iii. if said HCA sample is classified as an inflammatory HCA sample, then only following up said subject if HCA<5 cm, or performing surgical resection if HCA>5 cm;
iv. if said HCA sample is classified as a β catenin mutated HCA sample, then performing surgical resection whatever the HCA size.
